(19) United States
(12) Patent Application Publication
(10) Pub. No.: US 2009/0318891 A1
(43) Pub. Date: Dec. 24, 2009

(54) Delivery device, system, and method for delivering substances into blood vessels

(76) Inventors: Ronald Marcotte, New Gloucester, ME (US); Walter Hebold, Casco, ME (US); Milton Waner, New York, NY (US); Louis Fink, Las Vegas, NV (US); Mark Cowperthwaite, Naples, ME (US)

Correspondence Address: Lawson & Persson, P.C., P.O. Box 712, Laconia, NH, 03247, US

(21) Appl. No.: 11/398,507

(22) Filed: Apr. 5, 2006

(51) Int. Cl. A61M 5/145; A61M 5/14

(52) U.S. Cl. 604/510; 604/131; 604/151

(57) ABSTRACT

A delivery device delivery system and a method for delivering substances into blood vessels. The delivery device includes a body having a first end, a second end, and an outer surface. A first substance reservoir is disposed within the body and at least one cannula extends from the first end of the body. The cannula includes a cannula tip having a cannula opening therethrough and a cannula sheathing defining an interior passage in fluid communication with the first substance reservoir. A means is provided for delivering the first substance from the first substance reservoir through one of the at least one cannula, and a means is provided for delivering the second substance through one of the at least one cannula.

### FIELD OF THE INVENTION

The present invention relates to the delivery of substances, such as dyes, into subcutaneous blood vessels. In particular, the present invention relates to improved delivery devices, systems and methods for delivering substances into blood vessels and observing the flow of these substances to verify proper delivery thereof.

### BACKGROUND OF THE INVENTION

Medical treatment errors are increasingly recognized as an aspect of healthcare that needs greater attention. A recent report from the Institute of Medicine concluded that medical errors kill from 44,000 to 98,000 hospitalized Americans each year. Errors in drug delivery or drug dosages are all too common in medical practice and such errors are responsible for a significant share of these deaths. Consequently, there is a need for improved systems and procedures that verify that drugs are properly delivered.

Successful IV drug delivery depends on medical practitioners properly placing the IV needle or catheter inside the appropriate vessel such that the drug flows to the intended location. This is especially important in the administration of, for example, drugs used in chemotherapy, which are highly toxic. In such cases, it is of great importance that medical practitioners are able to avoid inadvertently perforating blood vessel walls during IV access, or injecting these drugs into the wrong vessels, as the failure to deliver these hazardous agents correctly to the proper location within the patient can lead to patient injury or even death.

Another instance in which proper drug delivery is critical is the performance of certain direct-puncture interventional radiology procedures, in which highly toxic drugs must be delivered to less-prominent vessels in and around the face and neck. Locating these smaller blood vessels can be a challenging task that requires years of practice and experience. Further complicating matters, the direction of blood flow within these vessels is not always evident, yet is critically important. If toxic drugs are introduced to vessels in which blood flows toward the brain, the damage to the brain could severely harm or kill the patient. Therefore, it is important that medical practitioners have a means to verify the direction of blood flow within vessels into which they are introducing irritant drugs, before the drug is delivered.

In order to help reduce the risk of incorrect drug delivery, verification techniques have been developed in which benign substances, such as dyes, that are visible using x-ray, CT, or magnetic resonant imaging, are injected into the target blood vessel, prior to the injection of the therapeutic drug. The flow direction and destination of the substance is then monitored through a series of image exposures in order to verify that the drug, when delivered, will travel to its intended location. Unfortunately, these imaging techniques are slow and expensive and, in the case of x-ray imaging, subject patients to excessive radiation exposure.

Another drawback of traditional dye based diagnostic systems is the difficulty in quickly and accurately identifying the target blood vessel(s) and gaining IV access with a minimum of physical and emotional trauma to the patient. Medical practitioners encounter difficulty in gaining IV access in a significant portion of the patient population for which subsurface blood vessels are obscured. Such patients include obese patients, darkly pigmented patients, neonates (infants from birth to four weeks of age), children under four years of age, patients experiencing lowered blood pressure, and patients who have collapsed veins. This difficulty is further exacerbated in cases in which substances must be introduced into less prominent blood vessels as these less prominent blood vessels cannot be found easily by visual and tactile clues, and accessing them may require multiple sticks to the patient, which thereby causes the patient physical and emotional pain and trauma. Inhibited IV access and diagnostic procedures can also subject medical practitioners to legal liability risk, by contributing to the complications associated with improper, ineffective, or delayed IV access and diagnosis.

In cases where multiple injections must be made, the time required to find blood vessels, inject substances, transport patients to imaging equipment, take, develop and evaluate medical images, make injections, remove, and either flush or discard catheters for each injection, is especially cumbersome. In these circumstances, the need to verify proper placement of each injection delays medical treatment unnecessarily, vastly increases treatment costs, increases patient stress, and further jeopardizes patient health.

Therefore, there is a need for an improved system and method that is capable of verifying that a drug is correctly delivered, that allows blood vessels to be accurately and rapidly located even under difficult conditions and body types (e.g., obese patients, dark pigmentation skin, neonates, collapsed veins, low lighting), that reduces patient pain and trauma, both emotionally and physically, that does not require the use of expensive and potentially hazardous x-ray or magnetic resonance imaging devices to provide such verification, that greatly reduces the time and expense required to safely perform multiple injections, and that allows minimally trained medical staff to verify that a drug is correctly delivered.

### SUMMARY OF THE INVENTION

The present invention is a delivery device for delivering a first substance and a second substance into a blood vessel, a delivery system for accurately delivering a substance into a blood vessel, and a method for delivering a therapeutic substance into blood vessels using a delivery device and observing the flow of an IR-visible substance with the aid of an infrared imaging system to verify proper delivery of the therapeutic substance.

In its most basic form, the delivery device for delivering a first substance and a second substance into a blood vessel includes a body having a first end, a second end, an outer surface. A first substance reservoir disposed within the body. At least one cannula extends from the first end of the body. The cannula includes a cannula tip having a cannula opening therethrough and a cannula sheathing defining an interior passage in fluid communication with the first substance reservoir. A means is provided for delivering the first substance from the first substance reservoir through one of the at least one cannula; and a means is provided for delivering the second substance through one of the at least one cannula.

In one embodiment of the delivery device, the means for delivering the second substance is a drug port extending from the outside surface of the body. The drug port is dimensioned to allow passage of a hypodermic needle therethrough and is in fluid communication with the cannula such that the substance may be delivered from the hypodermic needle through the drug port and the cannula. In some such embodiments, the first substance reservoir is a substantially cylindrical bore extending into the body from the second end of the body, and the means for delivering the first substance from the first substance reservoir through the cannula is a plunger dimensioned to mate with the cylindrical bore and push the first substance disposed within the first substance reservoir through the cannula. The plunger preferably includes a through-hole dimensioned to allow a catheter needle to be disposed therethrough and a sealing means for sealing the plunger about the catheter needle such that the first substance cannot leak through the through-hole when the catheter needle is disposed therethrough. In such embodiments, the interior passage of the cannula is in concentric relation with the through-hole and is dimensioned to allow the catheter needle to be inserted therein, and the cannula opening of the cannula tip is dimensioned to prevent passage of the catheter needle therethrough.

In other embodiments of the delivery device, the first substance reservoir is a hollow portion of the body and the means for delivering the first substance from the first substance reservoir through the cannula is a pump actuator extending from the second end of the body. A one-way valve is in communication with the pump actuator and an internal bladder is disposed within the first substance reservoir proximate the second end of the body. In this arrangement, the pump actuator is adapted to pump air through the one-way valve to expand the internal bladder such that the first substance is forced from the substance reservoir through the cannula.

In other embodiments of the delivery device, the first substance reservoir is a hollow portion of the body and the means for delivering the first substance from the first substance reservoir through the cannula is a pump actuator extending from the second end of the body, a one-way valve in communication with the pump actuator, and an internal bladder disposed within the first substance reservoir proximate the second end of the body. In such embodiments, the pump actuator is preferably adapted to pump air through the one-way valve to inflate internal bladder such that the first substance is forced from the first substance reservoir through the cannula.

In other embodiments of the delivery device, the first substance reservoir is a substantially cylindrical bore extending into the body from the second end of the body and the means for delivering the first substance from the first substance reservoir through the cannula is a first plunger dimensioned to mate with the cylindrical bore and push the first substance disposed within the first substance reservoir through the cannula. In some such embodiments, the means for delivering a second substance through the cannula is a second substance reservoir disposed within the first plunger and in fluid communication with one of the at least one cannula, and a means for delivering the second substance from the second substance reservoir through one of the at least one cannula. In some such embodiments, a flexible tube is provided in fluid communication with the second substance reservoir and the cannula, the second substance reservoir includes a substantially cylindrical bore extending into the first plunger and the means for delivering the second substance from the first substance reservoir through one of the at least one cannula is a second plunger dimensioned to mate with the cylindrical bore and push the second substance disposed within the second substance reservoir through the flexible tube and said cannula.

The preferred embodiment of the delivery device includes a safety means for preventing one of the first substance and the second substance from being delivered through the cannula before another of the first substance and the second substance has been delivered through the cannula.

Still other embodiments of the delivery device include a second substance reservoir in fluid communication with one of the at least one cannula. In some such embodiments, the first substance reservoir includes a tube filled with the first substance, the second substance reservoir includes a tube filled with the second substance, the body includes at least two mating bores in which the first substance reservoir and the second substance reservoir are disposed and secured. In these embodiments, it is preferred that the means for delivering a first substance from the first substance reservoir through the cannula is a first selector disposed upon the body and adapted to control the delivery of the first substance from the first substance reservoir through the cannula and that the means for delivering a second substance through one of the at least one cannula is a second selector disposed upon the body and adapted to control the delivery the second substance from the second substance reservoir through the at least one cannula. In some such embodiments, the first substance reservoir is a pressurized tube filled with the first substance and the second substance reservoir is a pressurized tube filled with the second substance.

In some embodiments of the delivery device, at least one actuator is adapted to deliver at least one of the first substance and the second substance through the at least one cannula, the means for delivering a first substance from the first substance reservoir through the cannula is at least one actuator and the means for delivering a first substance from the second substance reservoir through the cannula is one of the at least one actuator. In embodiments utilizing at least one actuator, it is preferred that there be a first actuator and a second actuator. In such embodiments, the means for delivering the first substance from the first substance reservoir through the cannula is the first actuator in communication with the first substance reservoir and the means for delivering the second substance from the second substance reservoir through the cannula is the second actuator.

In still other such embodiments, the delivery device includes a third substance reservoir in fluid communication with one of the at least one cannula and the third substance reservoir comprises a tube filled with a third substance. In these embodiments, the body includes at least three mating bores in which the first substance reservoir, the second substance reservoir, and the third substance reservoir are disposed and secured, and at least three selectors adapted to control the delivery of the first substance from the first substance reservoir through the at least one cannula, the second substance from the second substance reservoir through the at least one cannula, and the second substance from the second substance reservoir through the at least one cannula. In a preferred embodiment, the first substance reservoir is an IR-visible substance reservoir filled with an IR-visible substance, the second substance reservoir is a drug reservoir filled with a drug, the third reservoir is a flushing reservoir filled with a flushing substance, and the device includes a safety means for controlling the operation of the selectors such that the drug may not be delivered before a first amount of the IR-visible substance has been delivered, and such that second amount of the IR-visible substance may not be delivered until the flushing substance has been delivered through the cannula. In other embodiments, the third reservoir is filled with another drug, or solution of multiple drugs instead of the flushing substance, while still other embodiments utilize more than three reservoirs, each of which may be filled with drugs, combinations of drugs, and/or flushing substances.

Still other embodiments of the delivery device include a first cannula and a second cannula. In these embodiments, it is preferred that the first substance reservoir is in fluid communication with the first cannula and the means for delivering a second substance through one of the at least one cannula is in fluid communication with the second cannula. In some such embodiments, the means for delivering a second substance through the cannula includes a second substance reservoir in fluid communication with the second cannula and a second actuator in communication with a second substance reservoir.

Finally, in some embodiments of the delivery device, the means for delivering the first substance from the first substance reservoir through the cannula is a means for selectively delivering a desired amount of the first substance from the first substance reservoir through the cannula. The preferred means for selectively delivering a desired amount of the first substance from the first substance reservoir through the cannula includes a substantially flexible tube, a means for collapsing a portion of the substantially flexible tube and a means for moving the means for collapsing a portion of the substantially flexible tube toward the cannula.

In its most basic form, the delivery system for accurately delivering a substance into a blood vessel includes one of the embodiments of the delivery device described above in combination with an imaging system. The imaging system includes at least one infrared emitter configured to illuminate a region under a surface of skin with waves of infrared light, an infrared detector configured to accept waves of infrared light reflected from the region under the surface of the skin, the infrared detector having an output for outputting a signal corresponding to image data, a computing unit having an input for accepting the image data from the infrared detector, and an output for outputting images corresponding to the image data, a display device for inputting the images from the output of the computing unit and displaying the images, and a power source in electrical communication with the infrared emitter, the infrared detector, the computing unit and the display device. In operation a user disposes the cannula of the delivery device within a blood vessel located beneath the surface of the skin, delivers the IR-visible substance into the blood vessel, views images of the IR-visible substance on the display of the imaging system to examine a flow pattern of the IR-visible substance and verify that the at least one cannula is properly disposed within a desired blood vessel, and delivers the second substance through one of the at least one cannula into the blood vessel.

In some embodiments of the delivery system, at least one substance for enhancing a visibility of the cannula by the imaging system, when compared with a visibility of the cannula without the substance disposed thereon, is disposed upon the cannula tip and the cannula sheathing.

In the preferred embodiment of the delivery system, the computing unit of the imaging system further includes a memory and means for enhancing and outputting result images in which enhanced images of blood vessels are shown within images of the region under the surface of the skin, and the images corresponding to the image data are the result images. It is likewise preferred that the imaging system include a headset, to which the infrared emitter, the infrared detector, the computing unit, the display, and the power source are attached to the headset. In such embodiments, the display is preferrably disposed such that a user is able to view both the display and the surface of the skin without removing the headset. The infrared detector of the preferred imaging system is a CMOS camera adapted to generate digital data corresponding to the waves of infrared light reflected from the subcutaneous blood vessels located in the region under the surface of the skin. A camera lens is preferably disposed between the surface of the skin and the CMOS camera. The preferred display of the imaging system is at least LCD screen, while it is likewise preferred that an optical lens be disposed between the LCD screen and an eye of a user. The preferred computing unit includes a digital signal processing unit and a data input in communication with the digital signal processing unit through the interface.

In its most basic form, the method for delivering a therapeutic substance into blood vessels using a delivery device and observing the flow of an IR-visible substance with the aid of an infrared imaging system to verify proper delivery of the therapeutic substance includes the steps of preparing a body target area and supplying power from the power source to the infrared emitter, infrared detector, computing unit, and display of the imaging system, such that infrared light is emitted by the infrared emitter, reflected infrared light is received by the infrared detector and converted into signals sent to the computing unit, the computing unit accepts the signals and outputs image data to the display, and the display displays the images. The basic method also includes the steps of accessing a target blood vessel, introducing the IR-visible substance into the target blood vessel, locating the target blood vessel such that images of the target blood vessel are captured by the infrared detector and displayed on the display, examining a flow of the IR-visible substance through the target blood vessel by viewing the images of the target blood vessel on the display of the imaging system, determining whether the flow of the IR-visible substance flow is acceptable, and delivering the therapeutic substance into the target blood vessel.

In a preferred embodiment of the method, the step of examining flow patterns involves examining images displayed on the display to determine the presence of a leakage through the target blood vessel by observing the IR-visible substance flowing outside of the target blood vessel.

In another preferred embodiment of the method, the step of examining flow patterns comprises examining images displayed on the display to determine whether the IR-visible substance flows in an intended direction within the target blood vessel.

In another preferred embodiment of the method, the step of examining flow patterns comprises examining images displayed on the display to determine whether and the IR-visible substance flows to the proper destination within the patient's bloodstream.

In still another preferred embodiment of the method, the computing unit of the imaging system enhances images of the target blood vessel before outputting the images to the display, the locating step is performed before the accessing step, and the accessing step includes the step of viewing an enhanced image of the target blood vessel on the display of the imaging system and piercing the target blood vessel with the aid of the enhanced image. In such embodiments, it is preferred that the locating step includes the steps of directing incident light from the infrared emitters on a target area of a surface of a skin and viewing the enhanced image of blood vessels located beneath the target area on the display. In embodiments where the display of the imaging system includes an optical lens disposed between the display and an eye of a user, the locating step preferably includes the steps of viewing the unenhanced image on the target area of the skin, and adjusting the optical lens to correct the enhanced image displayed on the display for depth perception differences between the enhanced image and the unenhanced image. In still other embodiments, the step of locating a target blood vessel includes the steps of viewing the unenhanced image on the target area of the skin and adjusting the display to correct the enhanced image displayed on display for depth perception differences between the enhanced image and the unenhanced image.

In embodiments in which the computing unit includes a digital signal processor and a memory and the imaging system comprises a data input, the method preferably includes the step of optimizing the imaging system using the data input to specify an enhancement algorithm stored in memory to be used by the digital signal processor to generate the enhanced image. This optimizing step preferably includes the step of selecting an enhancement algorithm based upon a factor selected from a group consisting of a body type, pigmentation, age of the patient, and characteristics of the IR-visible substance introduced into the target blood vessel. In other embodiments, the optimizing step includes using the data input to adjust at least one of an intensity level of the at least one infrared emitter and a wavelength of infrared light emitted by the at least one infrared emitter.

Finally, still other embodiments of the method include the step of flushing the interior passage of the cannula after the step of injecting the therapeutic substance into the blood vessel.

Therefore, it is an aspect of the invention to provide an improved system and method for verifying that a drug is correctly delivered.

It is a further aspect of the invention to provide an improved system and method for verifying that a drug is correctly delivered that increase the speed of such verification over current systems.

It is a further aspect of the invention to provide an improved system and method for verifying that a drug that greatly reduces the time and expense required to safely perform multiple injections.

It is a further aspect of the invention to provide an improved system and method for verifying that a drug is correctly delivered that reduces patients' physical and emotional pain and trauma associated with IV access verification.

It is a further aspect of the invention to provide an improved system and method for verifying that a drug is correctly delivered that does not require the use of expensive and potentially hazardous x-ray or magnetic resonance imaging devices to analyze flow patterns through the vessels.

It is a further aspect of the invention to provide an improved system and method for verifying that a drug is correctly delivered that it is effective at verifying that a drug is correctly delivered into less prominent blood vessels.

It is a further aspect of the invention to provide an improved system and method for verifying that a drug is correctly delivered that allows a minimally trained medical practitioner to verify that a drug is correctly delivered.

It is a still further aspect of the invention to provide an improved system and method for verifying that a drug is correctly delivered that allows blood vessels to be located, and drug delivery verified, more easily in difficult conditions and body types (e.g., obese patients, dark pigmentation skin, neonates, collapsed veins, low lighting).

It is a further aspect of the invention to provide an injection device that allows both dyes and drugs to be delivered to multiple sites on a patient without discarding the needle between such delivery at each site.

It is a further aspect of the invention to provide an injection device that is may be made to include an interlock device that ensures the proper sequencing of dyes and/or drugs to avoid damage from the improper injection of a toxic substance into the wrong location, or in the wrong sequence.

These aspects of the invention are not meant to be exclusive and other features, aspects, and advantages of the present invention will be readily apparent to those of ordinary skill in the art when read in conjunction with the following description, appended claims and accompanying drawings.

### BRIEF DESCRIPTION OF THE DRAWINGS

FIG. 1 is a front isometric view of the preferred embodiment of the imaging system that forms a part of some embodiments of the delivery system of the present invention.

FIG. 2 is a rear isometric view of the preferred embodiment of the imaging system of the present invention.

FIG. 3 is an isometric view of the preferred embodiment of the imaging system worn on the head of a user.

FIG. 4 is a diagram illustrating the operation of one embodiment of the imaging system of the present invention.

FIG. 5A is an image of a human forearm showing unpolarized visible spectrum light reflected from the forearm and captured by a camera.

FIG. 5B is a raw image of the human forearm of FIG. 5A showing cross-polarized infrared spectrum light reflected from the forearm and captured by the CMOS camera of the preferred system of the present invention.

FIG. 5C is an enhanced image resulting from the operation of the imaging system on the raw image of the human forearm of FIG. 5B.

FIG. 6 is an exploded view of a conventional prior art catheter with the catheter needle withdrawn from the cannula.

FIG. 7 illustrates a conventional prior art cannula inserted into a subcutaneous blood vessel of a patient's arm.

FIG. 8 is an exploded view a modified catheter that forms one embodiment of the delivery device of the present invention.

FIG. 9A is a side view of one embodiment of the delivery device of the present invention.

FIG. 9B is a rear isometric view of the embodiment of the delivery device of FIG. 9A.

FIG. 10A is an isometric view of one embodiment of the delivery device of the present invention.

FIG. 10B. is a cut away side view of the delivery device of FIG. 10A.

FIG. 10C is a cut away top view of the delivery device of FIG. 10A.

FIG. 11A is an isometric view of another embodiment of the delivery device of the present invention held in the hand of a user.

FIG. 11B. is a rear view of the delivery device of FIG. 11A showing the bores within which substance tubes are inserted.

FIG. 11C is an isometric view of one embodiment of a substance tube that serves as a substance reservoir in the embodiment of FIG. 11A.

FIG. 12A is an isometric view of another embodiment of the delivery device of the present invention.

FIG. 12B is an isometric view of the delivery device of the delivery device of FIG. 12A held within the hand of a user.

FIG. 13 is a section view of one embodiment of a means for selectively delivering a desired amount of a substance through the cannula.

FIG. 14 is a section view of another embodiment of a means for selectively delivering a desired amount of a substance through the cannula.

FIG. 15 is a side view of one embodiment of the delivery system in which the delivery device and the imaging system are combined together.

FIG. 16 is a flow diagram of a preferred method for delivering a therapeutic substance into blood vessels using a delivery device and observing the flow of an IR-visible substance with the aid of an infrared imaging system to verify proper delivery of the therapeutic substance.

### DETAILED DESCRIPTION OF THE INVENTION

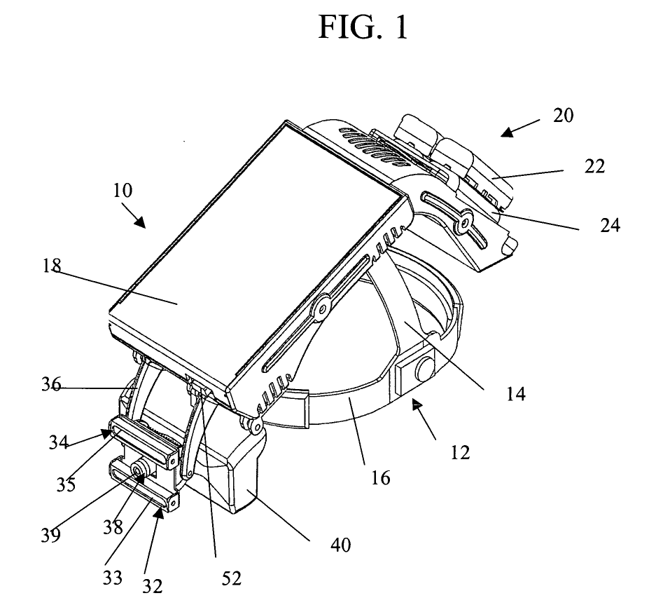

FIGS. 1-3 show the preferred embodiment of the imaging system 10 that forms a part of the delivery system of the present invention. The preferred embodiment of the imaging system 10 includes a headset 12 to which all system components are attached. The preferred headset 12 includes two plastic bands 14, 16; a vertical band 14 connected to sides of a horizontal band 16. The vertical band 14, holding most of the system components, generally acts as a load-bearing member, while the horizontal band 16 is adjustable such that it snugly fits about the forehead of the person using the system.

A pivoting housing 18 is attached to the headband 12. The housing 18 is substantially hollow and is sized to house and protect a headset electronics unit 120 disposed therein. Attached to the housing 18 are a power supply 20, an image capture assembly 30, and an enhanced image display unit 40.

The power supply 20 for the headset electronics unit 120 preferably includes two rechargeable lithium ion batteries 22, which are connected to the electronics unit via a pair of battery terminals 24 attached to the rear of the housing 18. The rechargeable lithium ion batteries 22 are preferably of a type commonly known as “smart batteries”, such as InfoLithium™ batteries manufactured by Sony Corp. of Osaka, Japan, which include an internal circuit that provides battery life feedback to the headset electronics unit 120. These batteries are commonly used with video camcorders and, thus, are readily available, are rechargeable without fear of memory problems, make the unit completely portable, and will provide sufficient power to the headset electronics unit 120 when two such batteries 22 are used. However, it is recognized that any power supply 20 known in the art to supply power to electronics, such as nickel cadmium batteries, nickel metal hydride batteries, alternating current power plugs, or the like, may be employed to achieve similar results.

The image capture assembly 30 is powered thorough the headset electronics unit 120 and includes a pair of infrared emitters 32, 34, and a camera 38, or other infrared detector, disposed between the infrared emitters 32, 34. The infrared emitters 32, 34 and camera 38 are preferably attached to a common mounting surface 31 and are pivotally connected to a pair of extension arms 36 that extend from the housing 18. Mounting in this manner is preferred as it allows the emitters 32, 34 and camera 38 to be aimed at the proper target, regardless of the height or posture of the person wearing the headset. However, it is recognized that both could be fixedly attached to the headset, provided the relationship between the emitters 32, 34 and camera 38 remained constant.

The infrared emitters 32, 34 of the preferred embodiment are surface mount LEDs (light emitting diodes) that feature a built-in micro reflector. Light emitting diodes are particularly convenient when positioned about the head because they are found to generate less heat then conventional bulbs and do not require frequent changing. Further, surface mount LED's that emit infrared light through light shaping diffusers to provide uniform light and are readily adapted for attachment to a variety of other flat filter media. The preferred infrared emitters 32, 34 each utilize a row, or array, of such LED's in front of which is disposed a light shaping diffuser (not shown). Such emitters 32, 34 may be purchased from Phoenix Electric Co., Ltd., Torrance, Calif. First polarizing filters 33, 35 are mounted in front to the light shaping diffusers of each of the infrared emitters 32, 34. These polarizing filters 33, 35 are preferably flexible linear near-infrared polarizing filters, type HR, available from the 3M Corporation of St. Paul, Minn. In operation, the LED's are powered through the headset electronics unit 120 and emit infrared light, which passes through the light shaping diffuser 205 and the first polarizing filters 33, 35 to produce the polarized infrared light 215 that is directed upon the object to be viewed.

The camera 38 is adapted to capture the infrared light 230 reflected off of the object to be viewed and to provide this “raw image data” to the headset electronics unit 120. The preferred camera 38 is a monochrome CMOS camera that includes a high pass filter (not shown) that filters out all light outside of the infrared spectrum, including visible light. A monochrome camera is preferred due to the superior contrast that it provides between blood vessels and the surrounding area. However, color cameras may be utilized in other embodiments, either with or without the inclusion of an integral filter. A CMOS camera is preferred as it produces pure digital video, rather than the analog video produced by the CCD cameras disclosed in the prior art, and is, therefore, not susceptible to losses, errors or time delays inherent in analog to digital conversion of the image. The CMOS camera may be any number of such cameras available on the market, including the OMNIVISION® model OV7120, 640×480 pixel CMOS camera, and the MOTOROLA® model XCM20014. In the test units, the OMNIVISION® camera was used with good success. However, it is believed that the MOTOROLA® camera will be preferred in production due to its enhanced sensitivity to infrared light and the increased sharpness of the raw image produced thereby.

A camera lens 240 is preferably disposed in front of the camera 38. This camera lens 240 is preferably an optical lens that provides an image focal length that is appropriate for detection by the camera 38, preferably between six inches and fourteen inches, eliminates all non-near IR light, and reduces interference from other light signals. The preferred camera lens 240 is not adjustable by the user. However, other embodiments of the invention include a camera lens 240 that may be adjusted by the user in order to magnify and/or sharpen the image received by the camera 38. Still others eschew the use of a separate camera lens 240 completely and rely upon the detection of unfocused light by the camera 38, or other infrared detector.

A second linear polarizing filter 39 is disposed in front of the lens 240 of the camera 38. This second polarizing filter 39 is preferably positioned so as to be perpendicular to the direction of polarization through the first polarizing filters 33, 35 in front of the infrared emitters 32, 34, effectively cross polarizing the light detected by the camera 38 to reduce spectral reflection. The polarizing filter 39 was selected for its high transmission of near-infrared light and high extinction of cross-polarized glare. Such polarizer may be purchased from Meadowlark Optics, Inc. of Frederick, Colo. under the trademark VERSALIGHT®.

The camera 38 is in communication with the headset electronics unit 120 and sends the raw image data to the unit for processing. The headset electronics unit includes the electronics required to supply power from the power supply 20 to the image capture assembly 30, and an enhanced image display unit 40, and the compatible digital processing unit 122 which accepts the raw image data from the camera 38, enhances the raw image, and sends an output of the enhanced image to the enhanced image display unit 40 and, optionally, to an interface 52. In the preferred embodiment, this interface 52 is standard VGA output 52. However, interface 52 may be any electronic data I/O interface capable of transmitting and receiving digital data to and from one or more input or output devices, such as an external monitor, external storage device, peripheral computer, or network communication path.

The preferred digital signal-processing unit 122 is a digital media evaluation kit produced by ATEME, Ltd SA, Paris, France under model number DMEK6414, which uses a Texas Instruments TMS320C6414 digital signal processor. This processing unit 122 is preferably programmed with an embodiment of the computer program means described in the applicants' co-pending U.S. patent application Ser. No. 10/760,051, in order to enhance the images. The image enhancement algorithms embodied in the computer program means utilize several elemental processing blocks, including (1) Gaussian Blurring a raw image with a kernel radius of 15, (2) adding the inverse Gaussian-blurred image to the raw image, and (3) level adjusting the result to use the entire dynamic range. Image enhancement is performed in a series of steps, which are coded into a computer program that runs on digital signal processor 120. The programming languages are typically C language and assembly language native to digital signal processor 120. An example algorithm is as follows:

ON device startup
BEGIN

Perform Initialization of Blur Kernel

END

WHILE device = ON

BEGIN

Acquire digital image data from the camera into RAM buffer
Save non-enhanced copy of the image data into another RAM
buffer
Perform 2D transform of image data in first RAM buffer into the
frequency domain
Perform smoothing of transformed image data USING Blur
Kernel
Perform 2D inverse transform of smoothed image data into the
spatial domain
Perform inversion of the smoothed image data
Perform add the inverted image data to the non-enhanced copy
of the image data
Perform contrast stretching
Perform gamma enhancement.
Send the enhanced image data to the display buffer

END

However, it is understood that other systems may use different means for similarly enhancing such images in near real-time and, therefore, it is understood that all embodiments of the invention need not include this program product or perform the methods described in the above referenced patent application.

The enhanced image is outputted from the processing unit to the enhanced image display unit 40. The preferred display unit 40 is distributed by i-O Display Systems of Sacramento, Calif., under the trademark I-Glasses VGA. This display unit 40 includes a binocular display that includes a pair of LCD screens in front of which are disposed a pair of optical lenses 42, 44 that allow the focal length to be adjusted for ease of viewing. The preferred an optical lenses 42, 44 provides image depth perception compensation to the user when the imaging system 10 is used in a bifocal mode. That is, when the user views the body target area via display 150, the optical lenses 42, 44 ensure that the image appears similarly sized and distanced as when the user views the target area without using display 40. However, it is understood that a monocular display unit 40 having no such focal length adjustment could likewise be used. The preferred display unit 40 also includes an on-screen display that is not currently used, but may be used in the future to show what enhancement option has been chosen by the user.

The imaging system 10 may be used in a total immersion mode, in which the user focuses on the target area by using exclusively display 40. Alternatively, the imaging system 10 may be used in a bifocal mode, in which the user views the body target area via a combination of display 40 and the naked eye. In bifocal mode, the user alternates between viewing the enhanced and non-enhanced image views of the body target area, by directing his/her gaze upward to display 40 or downward toward the body target area and away from display 150.

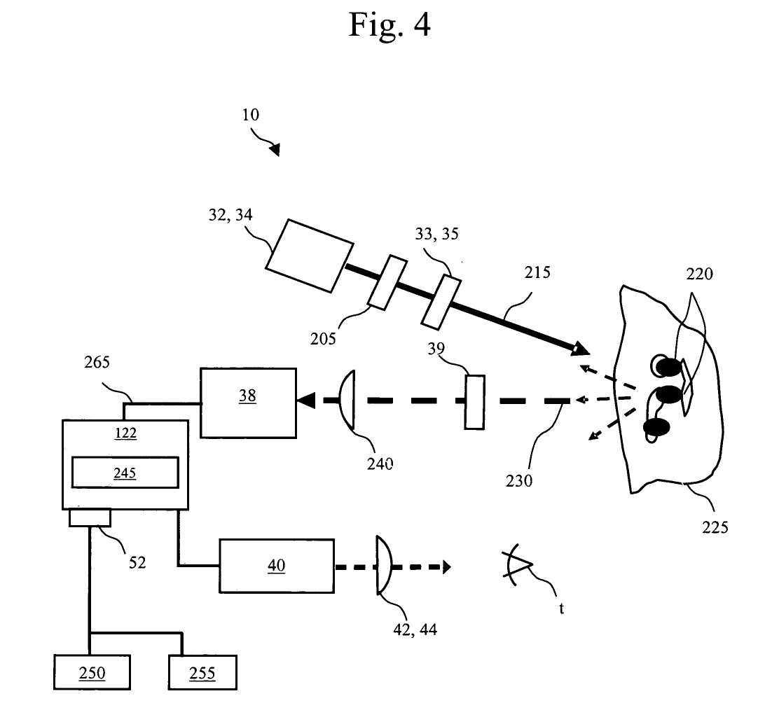

FIG. 4 illustrates one embodiment of the infrared imaging system 10 used to view subcutaneous blood vessels 220, such as arteries, veins, and capillary beds, which are present under the surface 225 of normal human skin. The infrared imaging system 10 described in connection with FIG. 4 includes all of the features of the preferred embodiment described above, in addition to including a camera lens 240, image data storage means 445, a data input 250, and data output 255.

Image data storage means 245 is any means of digital data storage that is compatible with digital signal processor 120 and may be used to store multiple enhanced and/or unenhanced images for future viewing. Examples of such image data storage are random access memory (RAM), read-only memory (ROM), personal computer memory card international association (PCMCIA) memory card, microdrives, compact flash memory, memory sticks, or other removable or fixed data storage means known in the art. Depending on memory size, hundreds or thousands of separate images may be stored on the image data storage means 245, either as still images, video clips, or a combination thereof.

Data output 250 is any external device upon which the image data produced by digital signal processor 120 may be viewed, stored, or further analyzed or conditioned. Examples of data output 250 devices include external video displays, external microprocessors, hard drives, and communication networks. Data output 250 interfaces with digital signal processor 120 via interface 52.

Data input 255 is any device through which the user of the imaging system 10 inputs data to digital signal processor 122 in selecting, for example, the appropriate enhancement algorithm, adjusting display parameters, and/or choosing lighting intensity levels. Examples of data input 255 devices include external keyboards, keypads, personal digital assistants (PDA), or a voice recognition system made up of hardware and software that allow data to be inputted without the use of the user's hands. Data input 255 may be an external device that interfaces with digital signal processor 120 via interface 52, or may be integrated directly into the computing unit.

Digital data path 265 is an electronic pathway through which an electronic signal is transmitted from the camera 38 to the digital signal processor 122.

In operation, the infrared imaging system 10 is powered on and the infrared emitters 32, 34 produce the necessary intensity of IR light, preferably at 850 nm to 950 nm wavelengths, required to interact and be absorbed by oxyhemoglobin and deoxyhemoglobin contained within normal blood, or at a different wavelength that may be required to interact with and reflect from, or be absorbed by, a substance being delivered into the blood vessel. The resulting light path passes through diffuser system 205, where it is dispersed into a beam of uniform incident light 215 of optimal intensity and wavelength. Incident light 215 passes through first polarizers 33, 35, which provide a first plane of polarization. Polarization of incident light 215 reduces the glare produced by visible light by reflection from skin surface 225. Incident light 215 is only partially absorbed by the oxyhemoglobin and deoxyhemoglobin that is contained with subcutaneous blood vessels 220 and/or the substance delivered into the blood vessel and, thus, produces reflected light 230.

Reflected light 230 passes through second polarizer 39, which provides a second plane of polarization. The second plane of polarization may be parallel, orthogonal, or incrementally adjusted to any rotational position, relative to the first plane of polarization provided by first polarizers 33, 35. Reflected light 230, passes through first lens 240, which provides an image focal length that is appropriate for detection by the camera 38, eliminates all non-near IR light, and reduces interference from other light signals.

Camera 38 detects reflected light 230 and converts it to an electronic digital signal by using CCD, CMOS, or other image detection technology. The resulting digital signal is transmitted to digital signal processor 122 via digital signal path 265. Digital signal processor 122 utilizes a number of algorithms to enhance the appearance of objects that have the spatial qualities of blood vessels, so that the user can distinguish blood vessels easily from other features when viewed on display 40. Such enhancement might include, for example, image amplification, filtering of visible light, and image analysis. The resulting digital signal is transmitted to display 40 via digital signal path 265, where it is rendered visible by LCD, CRT, or other display technology. Additionally, the resulting digital signal may be outputted to an external viewing, analysis, or storage device via interface 52. The image produced by display 40 is then corrected for depth perception by second lens 260, such that, when the user views the body target area via display 40, the image appears similarly sized and distanced as when the user views the target area with the naked eye.

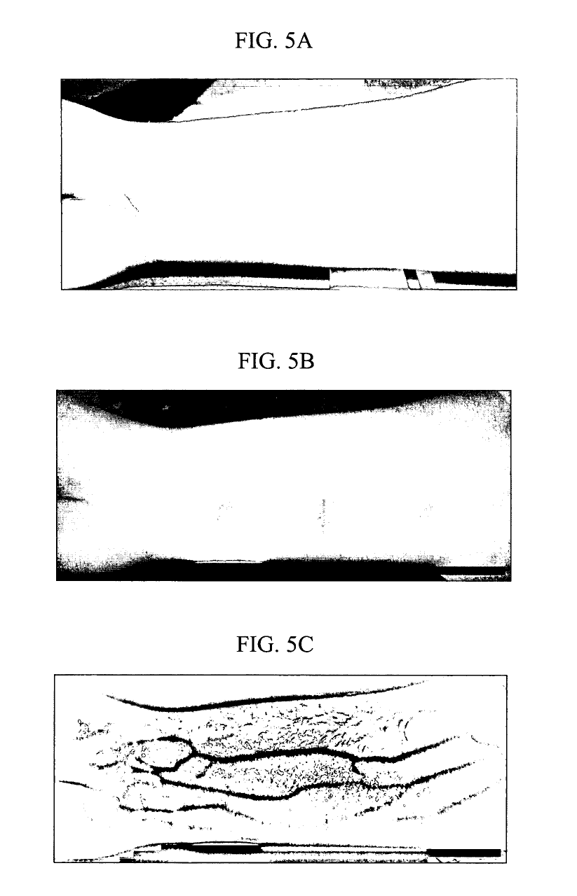

FIGS. 5A, 5B and 5C demonstrate the image enhancement produced by the system of the present invention. FIG. 5A is a photograph of a human forearm using light from the visible spectrum. As seen from this photograph, it is difficult to locate the veins upon visual inspection. FIG. 5B is a raw image of the same human forearm sent from the image capture assembly 30 of the present invention to the processing unit. The veins in this image are considerably more visible than those in FIG. 5A. However, they are not sufficiently dark and well defined to allow easy location of the veins during venepuncture. FIG. 5C is an enhanced image using the image enhancement process of the present invention. As can be seen from FIG. 5C, the veins are very dark and, therefore, are easily located for venepuncture.

It is noted that the imaging system 10 that forms part of the delivery system does not need to include all of the features of the preferred imaging system 10. Rather, the imaging system need only include at least one infrared emitter an infrared detector, a computing unit, a display device, and a power source. Therefore, the invention should not be seen as limited to delivery systems and methods utilizing the preferred imaging system 10 described in connection with FIGS. 1-5.

The delivery system of the present invention also includes a delivery device 200 for delivering substances into the blood vessel. As described in detail below, the delivery device 200 may take many forms, provided it is capable of delivering at least two different substances to the blood vessel without the need to withdraw the device after delivery of each substance and reinsert it in order to deliver the next substance.

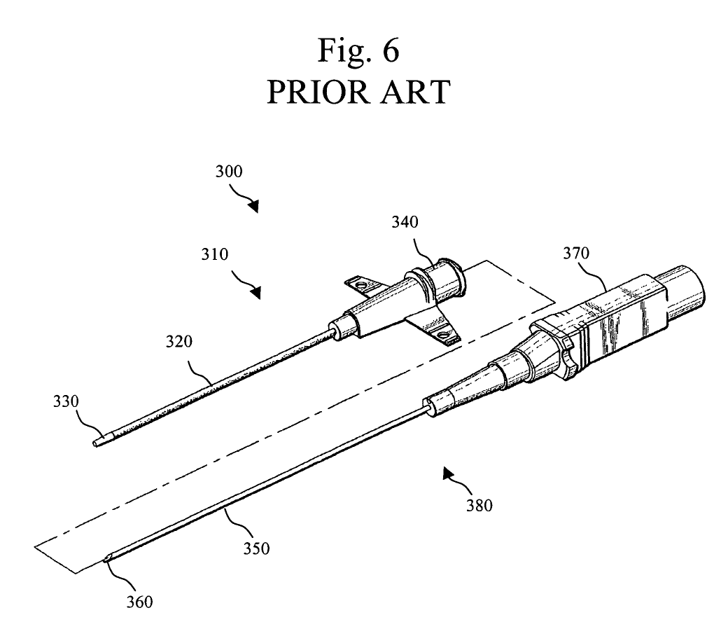

The delivery device 200 may be a catheter 300, such as an intraluminal, indwelling catheter, which is well known in standard medical practice and is presented in FIG. 6 for illustrative purposes. FIG. 6 shows an exploded view of a catheter 300, with the catheter needle 350 withdrawn from cannula 310. Catheter 300 includes a cannula 310, and a catheter body 380. Cannula 310 further includes a cannula sheathing 320, a cannula tip 330, and a cannula housing 340. Catheter body 380 further includes a catheter needle 350, a needle tip 360, and a flash chamber 370. An exploded view of a catheter is fully described and shown in US2004/0019280, US2003/0187360, and US2002/0115922, which are hereby incorporated by reference.

Cannula sheathing 320 is a hollow body that is constructed, typically, of medical-grade plastic and that has an inside diameter sufficient for receiving catheter needle 350. Catheter needle 350 is a hollow needle that is sheathed with cannula sheathing 320. Needle tip 360 is the sharp proximal tip of catheter needle 360 and protrudes from cannula tip 330 a sufficient distance in order to allow for piercing of the skin. The specific distance of penetration is based upon a number of factors, including the procedure to be performed, the body type of the patient and the user's personal preference. Accordingly, a sufficient distance in this context means a distance that the user deems to be sufficient. Cannula housing 340 may receive standard intravenous tubing (not shown) in an IV catheter. Flash chamber 370 is preferably constructed of medical-grade plastic and is a hollow chamber forming the distal end of catheter body 380.

An IR-opaque or IR-reflective substance or pattern may be applied to catheter needle 350 and needle tip 360, so as to render the needle position and travel path more visible to the medical practitioner when viewed with the imaging system 10 and, thus, assist in catheter placement. An IR-opaque substance, such as indocyanine green, may be applied to catheter needle 350 and needle tip 360. Alternatively, an IR-opaque or an IR-reflective pattern, such as solid bands, “zebra stripes,” or similar strongly identifiable markings may be applied to cannula sheathing 320. The intent is to produce a pattern that is easily visualized via display 40 of the imaging system 10 and that is distinctive from nearby anatomical structures. The IR-opaque or IR-reflective substance or pattern may be applied to catheter 300 during manufacture or sometime prior to patient treatment. Alternatively, catheter 300 and/or cannula tip 330 may be illuminated by IR radiation that is provided to catheter 300 via fiber optics, micro-diodes, or other IR-emitting source. These and additional examples of embodiments of catheter 300 are further disclosed in detail in U.S. patent applications US2004/0019280, US2003/0187360, and US2002/0115922.

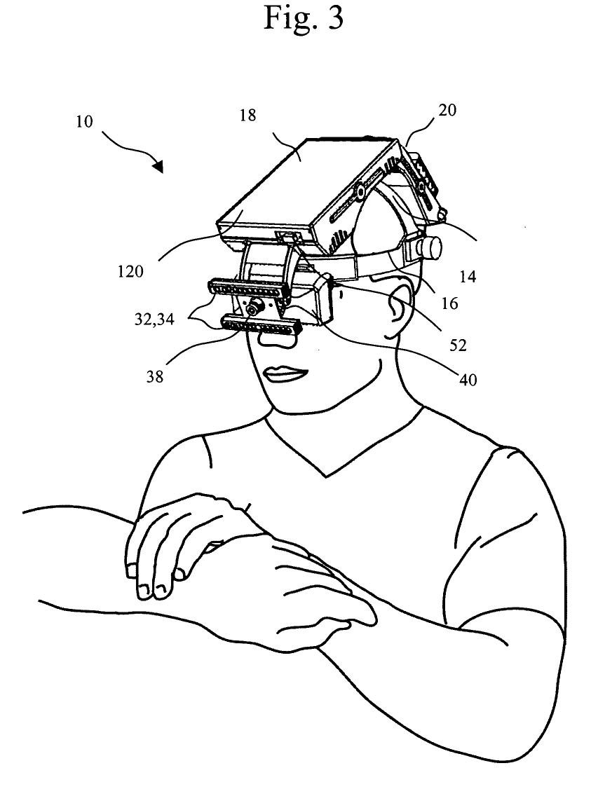

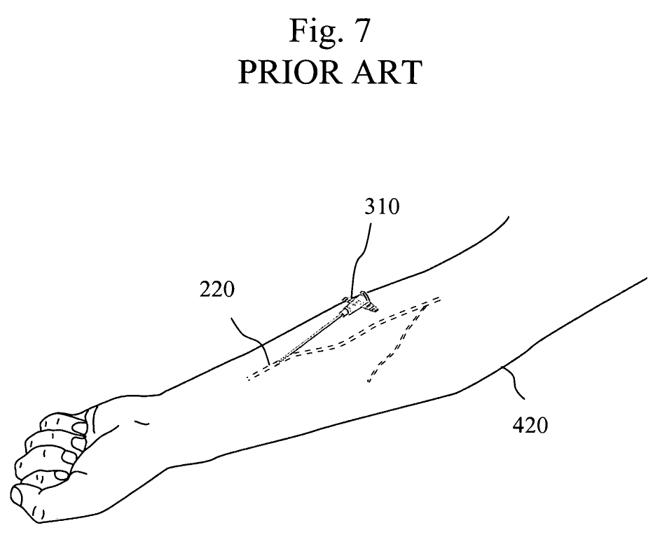

In delivery systems utilizing the preferred imaging system 10 and the catheter 300 of FIG. 6, a medical practitioner user prepares a patient's body target area for catheter 300 insertion by using standard medical practices, including, for example, cleaning the target area and applying a tourniquet. User 199 puts on the headset 12, provides power to the imaging system 10, and optimizes various parameters of system 10, including, for example, the patient's body type, body target area, and skin pigmentation. The user then locates the target blood vessel in the manner described above with reference to FIG. 3 Once the target blood vessel is located, user looks downward from display 40 to view catheter 300 in his/her visual field. Utilizing either his/her naked eye or the IR-enhanced image that appears on display 40, the user aligns catheter 300 above and parallel to the target blood vessel, pierces the skin surface with needle tip 360, and introduces the catheter 300 into the target blood vessel. When the catheter 300 enters the target blood vessel, blood will flow into flash chamber 370 alerting the user to its entry. Further, in cases an IR-opaque or IR-reflective substance or pattern are applied to cannula sheathing 320, the position and travel path of catheter needle 350 is clearly visible to user on display 40, which allows user to guide its depth and travel path more accurately and to provide a further visual indication that the blood vessel has been accessed. The user then advances catheter 300 into the target blood vessel until a sufficient depth has been reached, after which catheter needle 350 and catheter body 380 are withdrawn, which leaves cannula sheathing 320 remaining in the target blood vessel. Cannula 310 is secured in place, and the procedure is completed by use of standard medical practices. The result of such a procedure performed on a human forearm is shown in FIG. 7.

Once the cannula 310 is secured in place, an IR-visible substance, such as indocyanine green, is then introduced into cannula 340 by means of a standard hypodermic needle or IV line (not shown). The IR-visible substance flows from cannula housing 340, into cannula sheathing 320, out of cannula tip 330, and into the target subcutaneous blood vessel. Once the IR-visible substance enters the patient's blood stream, the medical practitioner monitors the flow by using the imaging system 10. Such monitory may include verifies the direction of flow and target location of the IR-visible substance. If the flow direction or target location is not correct, the medical practitioner repositions or relocates cannula 310 and repeats the verification procedure. Once the medical practitioner verifies the correct direction of flow of the IR-visible substance, the therapeutic drug is introduced into cannula 310 by means of a second hypodermic needle or IV line. Flow of the drug is then identical to that of the IR-visible substance.

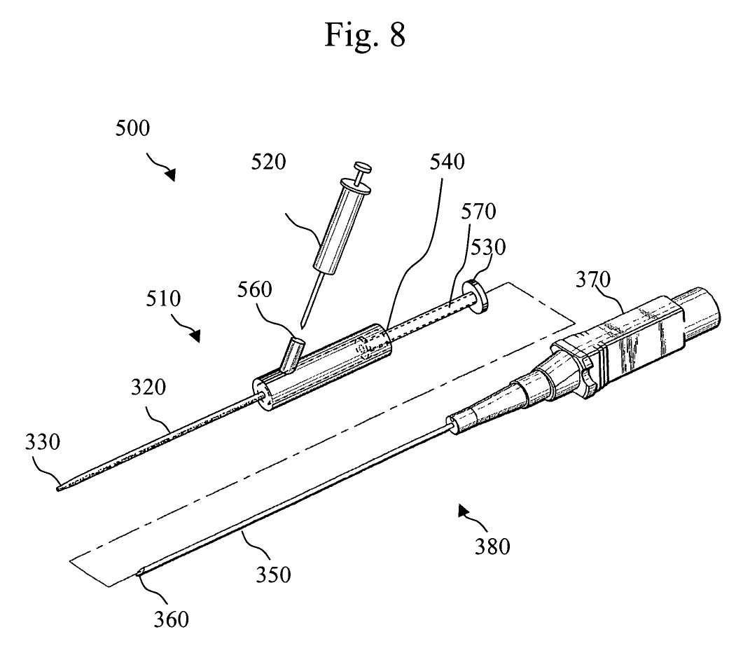

In other embodiments of the delivery system, the delivery device 200 is a modified catheter, such as the catheter 500 shown in FIG. 8. FIG. 8 illustrates one embodiment of a modified catheter 500 for injecting sequentially IR-visible substances and therapeutic drugs into a patient's blood vessels. Modified catheter 500 includes a modified cannula 510 and a drug hypodermic needle 520. Modified cannula 510 further includes a plunger 530, an IR-visible substance reservoir 540, a drug port 560, and a through-hole 570. Modified catheter 500 further includes cannula 310, cannula sheathing 320, cannula tip 330, catheter needle 350, needle tip 360, flash chamber 370, and catheter body 380, as described in reference to FIG. 6.

Plunger 530 is a pressure-sensitive plunger similar to that of a standard hypodermic syringe. Plunger 530 features an axial through-hole 570 that passes through the plunger shaft and is of sufficient inside diameter to allow the passage of catheter needle 350. Typically, plunger 530 is constructed of medical-grade plastic or other durable and disposable material. Sealing means, is preferably provided for sealing the plunger 530 about the catheter needle 350 such that the IR-visible substance cannot leak through the through-hole 570 when the catheter needle 350 is disposed therethrough. This sealing means is preferably a self-sealing membrane similar to those used in conventional drug ports.

IR-visible substance reservoir 540 is a hollow body and is, typically, constructed of medical-grade plastic and contains a dosage of an IR-visible substance appropriate to the treatment of a specific patient.

Cannula sheathing 320 of modified cannula 510 is a hollow body that is constructed, typically, of medical-grade plastic and is capable of being inserted into a patient's target blood vessel by means of catheter body 380 in a procedure similar to that of cannula 320 described with reference to FIGS. 6 and 7.

Drug port 560 contains a self-sealing membrane and is capable of receiving an injection of liquid drugs from drug hypodermic needle 520. Drug port 560 is integrated into IR-visible substance reservoir 540, such that drugs introduced into drug port 560 flow directly through IR-visible substance reservoir 540, through cannula sheathing 320, and into the patient's target blood vessel.

Drug hypodermic needle 520 is a conventional hypodermic needle designed to deliver liquid therapeutic substances into the bloodstream via drug port 560.

In operation, IR-visible substance reservoir 540 is filled with a predetermined dosage of IR-visible substance sufficient to confirm the correct direction of flow and target location within a blood vessel of a specific patient. Modified cannula 510 is inserted into the patient's target blood vessel, with the aid of the imaging system 10, and catheter body 380 is withdrawn from modified cannula 510, which leaves cannula sheathing 320 in the patient's target blood vessel as described with reference to FIGS. 6 and 7.

Plunger 530 is then depressed a sufficient amount to force the prepared volume of IR-visible substance out of IR-visible substance reservoir 540, through cannula sheathing 320, and into the patient's target blood vessel. Once the IR-visible substance enters the patient's bloodstream, the medical practitioner monitors the substance flow via display 40 of the imaging system 10, which thereby enables the verification of the direction of flow and target location of the IR-visible substance. If the flow direction and/or target location are incorrect, the medical practitioner withdraws modified cannula 510, repositions or relocates modified catheter 500, refills IR-visible substance reservoir 540, and repeats the verification procedure. Once the medical practitioner verifies the correct direction of flow of the IR-visible substance, the therapeutic drug is introduced into drug port 560 by means of drug hypodermic needle 520 where it flows through the cannula tip 330 into the blood vessel in the identical location and direction as that of the IR-visible substance injected before it.

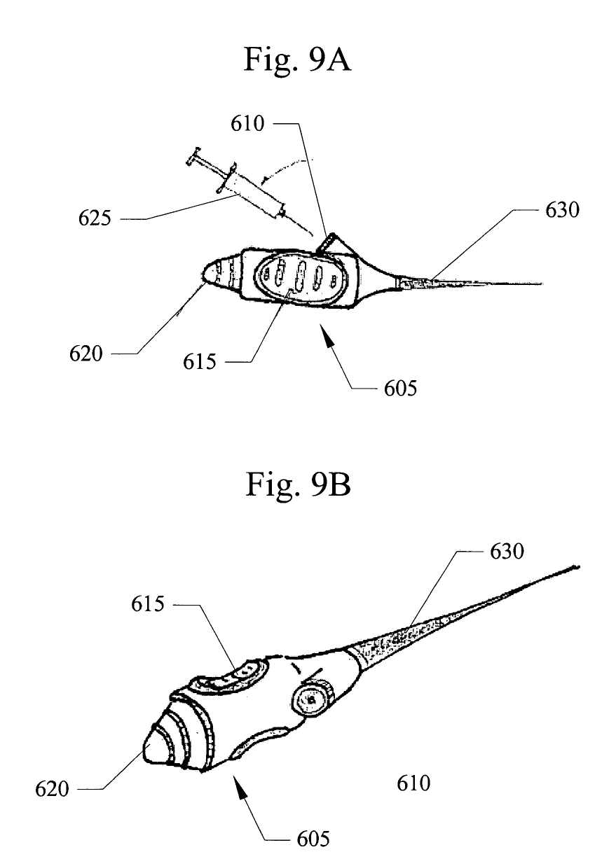

FIGS. 9A and 9B show an alternative delivery device 600 that allows both an IR-visible substance and a drug to be delivered into a blood vessel. The alternative delivery device 600 includes a substantially hollow body 605 having an actuator 620 that extends from one end and a cannula 630 that extends from an opposite end. The hollow interior of the body 605 forms an IR-visible substance reservoir, similar to the reservoir 540 of the embodiment of FIG. 8, which is filled with an IR-visible substance (not shown).

The IR-visible substance is delivered to a blood vessel through the cannula 630 by depressing the actuator 620. The actuator 620 may take many forms, including a plunger similar to the one described above. However, in the embodiment of FIGS. 9A and 9B, the actuator 620 is a pump actuator that includes a flexible membrane that pumps air through a one-way valve (not shown) to inflate an internal bladder (not shown) within the IR-visible substance reservoir in order to force the IR-visible substance from the reservoir through the cannula 630.

A drug port 610 is disposed through the side of the body 605 and is used to deliver a drug to the blood vessel after an examination of the flow pattern of the IR-visible substance verifies that the cannula is properly located and disposed within the blood vessel. The drug port 610 is preferably similar in all respects to the drug port 560 described with reference to FIG. 8, although drug ports 610 of different configurations may be substituted to achieve similar results.

The delivery device 600 is intended for insertion without the aid of a separate catheter and, therefore, the sides of the body 610 preferably includes gripping details 615 for ease of handling.

The delivery device 600 may be a single use device, or may be adapted for multiple uses. Such an adaptation may include a means, such as a threaded portion at the end of the body, for removing and replacing the cannula 630, and a means for refilling the reservoir with an IR-visible substance. Although other such variations would be readily apparent to those of ordinary skill in the art.

In embodiments of the delivery system utilizing the delivery device 600 of FIGS. 9A and 9B, the user will perform all of the same steps that were described above with reference the insertion of catheter 500 of FIG. 8, the examination of a flow of the IR-visible substance, and the delivery of the drug through the drug port 610 via a hypodermic needle. However, rather than removing the catheter needle 350 and body 380 and depressing the plunger 530, the user immediately delivers the IR-visible substance into the blood vessel after insertion of the cannula 630 by repeatedly depressing the actuator 620.

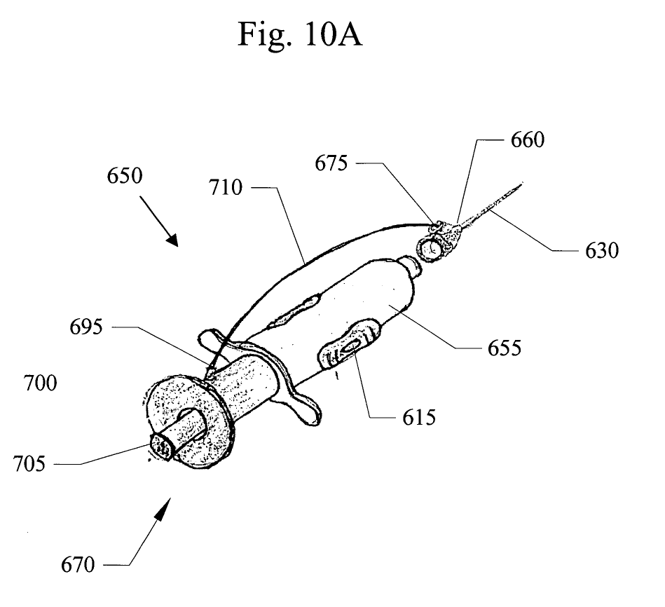

FIGS. 10A, 10B and 10C show still another embodiment of the delivery device 650 that includes a plunger assembly 670 that delivers both an IR-visible substance and a drug in a manner similar to a that of conventional syringe style hypodermic needle. In this embodiment the delivery device 650 includes a hollow body 655 having two open ends, a tip 660 attached to one end of the body 655, and a plunger assembly 670 disposed within the other open end of the body 655.

The body 655 of this embodiment may be a conventional syringe body made of a disposable medical grade plastic material. However, in the embodiment of FIGS. 10A, 10B and 10C, the sides of the body 610 preferably includes gripping details 615 for ease of handling the device during insertion.

The tip 660 is preferably a substantially hollow cone that includes a first IR-visible substance port 675 and a cannula 630 that extends therefrom. The tip 660 is preferably manufactured of a medical grade plastic and is preferably removably attached to the body to allow the body 655 and plunger assembly 670 of the delivery device 650 to be used multiple times.

The plunger assembly 670 includes a drug plunger 680, which fits within the hollow body 655 and operates in a manner identical to that of a conventional hypodermic needle syringe. However, the drug plunger 680 is different from those typically found in hypodermic needle syringes insofar as it includes a hollow reservoir portion 685 within which is disposed a IR-visible substance plunger 690 and a second IR-visible substance port 695 extending from the outside of the plunger 680 proximate to the handle 700 and in communication with the reservoir portion 685.

The IR-visible substance plunger 690 includes a smaller handle 705 that extends from the handle 700 of the drug plunger 680. Depressing the handle 705 causes the IR-visible substance plunger 690 to advance within the reservoir portion 685, pushing the IR-visible substance disposed therein through the second IR-visible substance port 695, where it passes through a flexible tube 710 and into the first IR-visible substance port 675, where it is delivered to the blood vessel through the cannula 630 that extends therefrom. In some embodiments, the drug plunger 680 includes a safety feature that prevents the drug plunger from being depressed until the IR-visible substance plunger 690 has been fully depressed, while others merely rely upon the skill of the user to prevent premature depression of the drug plunger.

In embodiments of the delivery system utilizing the delivery device 650 of FIGS. 10A, 10B and 10C, the user will perform all of the same steps that were described above with reference the insertion of delivery device of FIGS. 9A and 9B, the immediate delivery of the IR-visible substance into the blood vessel after insertion of the cannula, and the examination of a flow of the IR-visible substance. However, in this embodiment, the delivery of the IR-visible substance is accomplished by depressing the handle 705 of the IR-visible substance plunger 690 while the delivery of the drug is accomplished by depressing the handle 700 of the drug plunger 680 rather than through the insertion of a separate hypodermic needle into a drug port.

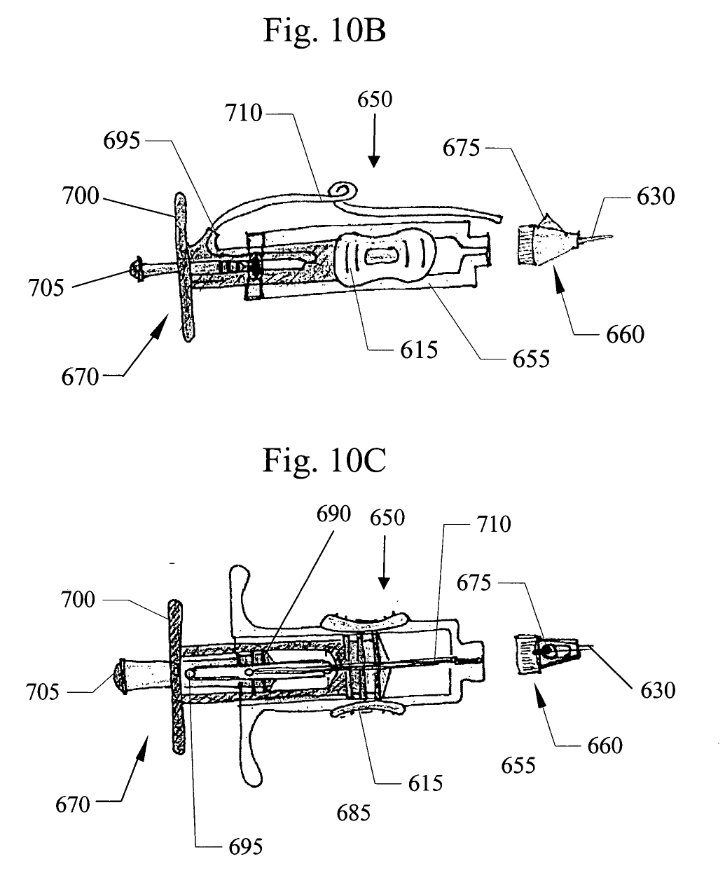

FIGS. 11A-11C show still another embodiment of the delivery device 720. In this embodiment, the delivery device 720 includes a body 725 having a plurality of selectors 727, 728, 729, 730 that allow three different substances to be selectively delivered through the cannula 310.

In the embodiment of FIGS. 11A-11C, the substances are disposed within individual pressurized tubes 733, 735, 737, which are secured within the end 739 of body 725 opposite the end 741 from which the cannula 310 extends and serve as the substance reservoirs for the device 720. As shown in FIGS. 11B and 11C, the pressurized tubes 733, 735, 737 are threaded into mating threaded bores 743, 745, 747 disposed within the end 741 of the body 725. Each of the pressurized tubes 733, 735 737 includes a valve stem 741, or other art recognized means for controlling the discharge of a pressurized fluid, that prevents discharge of the contents of the tubes 733, 735, 737 during storage but allows the contents to be discharged when the tubes 733, 735, 737 are threaded into mating threaded bores 743, 745, 747

The mating threaded bores 743, 745, 747 are each in communication with the selectors 727, 728, 729, 730, which control the position of a valve opening (not shown). Depending upon which of the selectors 727, 728, 729, 730, or combination thereof, that has been engaged, the valve opening is positioned such that it seals the pressurized tubes 733, 735, 737 from the cannula 310 or allows the contents of one of the pressurized tubes 733, 735, 737 to flow through the cannula.

In one embodiment of the invention, the pressurized tubes 733, 735, 737 are filled with an IR-visible substance, a drug, and a flushing medium, such as compressed air, nitrogen, or another inert gas. In this embodiment, selector 727 prevents discharge from any of the tubes 733, 735, 737, selector 728 allows the IR-visible substance to be discharged from tube 733, selector 729 allows the drug to be discharged from tube 735, and selector 737 allows the flushing medium to be discharged from tube 737. It is preferred that the selectors 727, 728, 729, 730 of this embodiment also include a safety feature that only allows them to be engaged in a specific order; i.e. selector 728 would not be engaged until after selector 727 has been engaged, selector 729 would not be engaged until after selector 728 has been engaged, selector 730 would not be engaged until after selector 729 has been engaged, and the unit could not be reset for another use until selector 730 has been engaged. However, such a safety feature is not required in order for this embodiment to be operational.

In embodiments of the delivery system utilizing the delivery device 720 of FIGS. 11A, 11B and 11C, the user will perform all of the same steps that were described above with reference to the device of FIGS. 10A, 10B and 10C, except that the delivery of the IR-visible substance and the delivery of the drug are accomplished by depressing selectors 728, 729 respectively, and the performance of the additional step of flushing the cannula 310 by depressing selector 730 after the drug has been delivered to the blood vessel.

Although pressurized tubes 733, 735, 737 have been shown and described in connection with delivery device 720 of FIGS. 11A, 11B and 11C, it is recognized that the pressurized tubes 733, 735, 737 may be replaced by flexible bladders. In such a variation, the bladders are each disposed within the body 725 and are pressurized by inflating an air bladder that exerts pressure on the bladders containing the desired substances. This may be accomplished in a manner similar to that described with reference to FIGS. 9A and 9B, or by other art recognized means from discharging a fluid from a flexible bladder. In a preferred embodiment utilizing flexible bladders, one bladder is filled with an IR-visible substance, another bladder is filled with a drug, and the third bladder is inflatable such that it acts both as the inflation bladder for exerting pressure on the other bladders and is in contact with the selector such that the pressurized gas may serve as a flushing medium to flush the cannula of any residual drug that is left therein after delivery into the blood vessel.

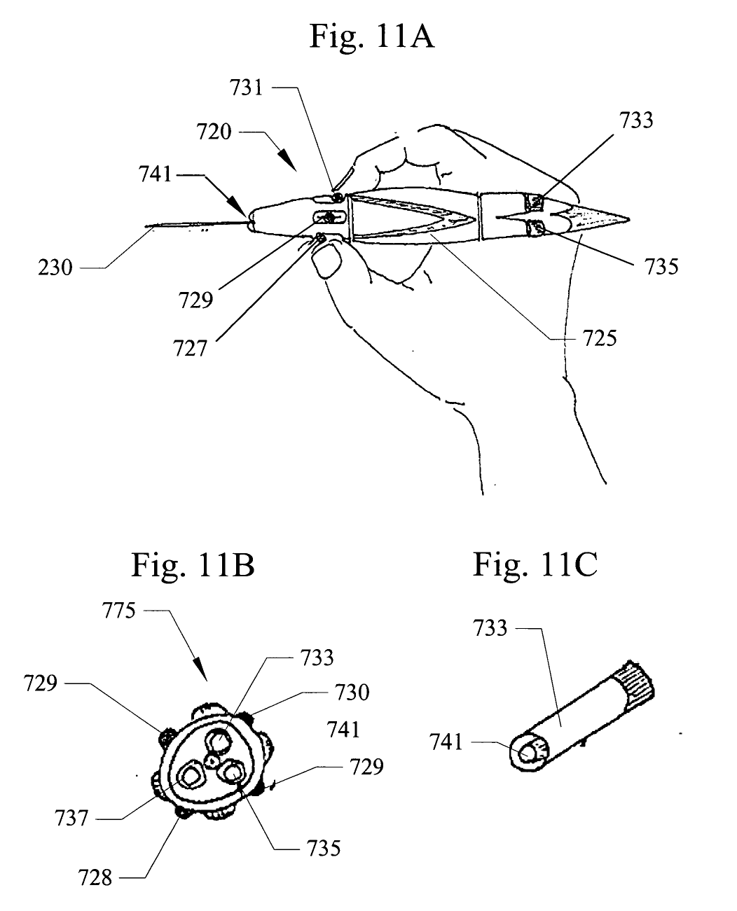

Referring now to FIGS. 12A and 12B, still another embodiment of the delivery device 800 is shown. In this embodiment, the delivery device 800 includes a body 805 that is ergonomically designed to fit within a user's hand, a cannula 310 that extends from the body 805, and a pair of actuators 807, 808 that may be depressed by the user to separately deliver two separate substances. The substances, preferably and IR-visible substance and a drug, may be arranged within the body 805 in any of the manners described herein and the actuators 807, 808 are specifically adapted to dispense the substances from its stored state. As was the case with other embodiments described herein, the actuators 807, 808 preferably include a safety feature that prevents the drug from being delivered before the IR-visible substance has been delivered.

In the performance of procedures involving multiple injections, it is preferable that the IR-visible substance and the drug not be completely dispensed during each injection cycle. Rather it is preferable that a small amount of the IR-visible substance and a small amount of drug be dispensed into one blood vessel, the cannula removed, and a the cycle immediately repeated in another blood vessel. In the embodiments described above using a single cannula 310, this is possible only if the interior of the cannula 310 is flushed between uses to prevent the delivery of residual amounts of the drug within the cannula 310 before verification of proper insertion. However, this flushing step may be eliminated by utilizing a multiple needle delivery device.

One embodiment of a multi-needle delivery device is a further modification the modified catheter described in connection with FIG. 8, in which the IR-visible substance reservoir 540 and plunger 530 are eliminated from the cannula housing 340 and, instead, replace the flash chamber 370 of the catheter 380. In operation, the catheter needle 350 is extended through the cannula sheathing 320 and inserted into the blood vessel in a manner similar to that described above. However, rather than disengaging the catheter body 380 from the cannula housing 340 and withdrawing the catheter needle 350, they remain attached together and a portion of the IR-visible substance disposed within the IR-visible substance reservoir 540 is injected through the catheter needle 350 and into the blood vessel by moving the plunger 530 forward a desired distance and then stopping. Once proper insertion has been verified using the techniques described above, a dose of the drug is injected through the drug port 560 and into the space formed between the outside of the catheter needle 350 and the inside of the cannula sheathing by advancing the plunger of the syringe type a hypodermic needle 520 a desired distance and then stopping. The delivery device 850 may then be removed and inserted in a different blood vessel, where the process is repeated. Because the IR-visible substance and the drug are segregated from one another by the catheter needle 350, and because neither will flow from the cannula tip 330 or needle tip 360 without advancing the plunger 530 or syringe, there is no need to perform a flushing step between injections. The concept of using multiple needles to segregate the IR-visible substance from the drug is not limited to variations of the catheter described in FIG. 8 and may be applied to any of the embodiments of the delivery device described herein.

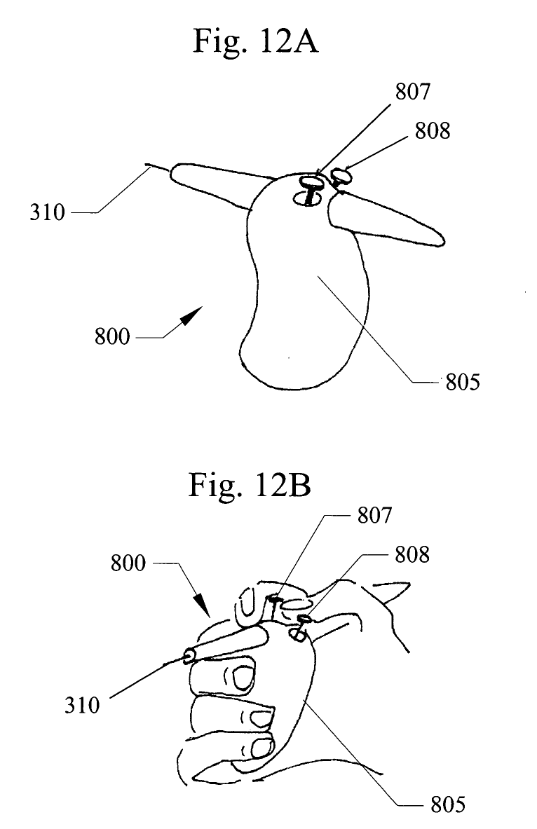

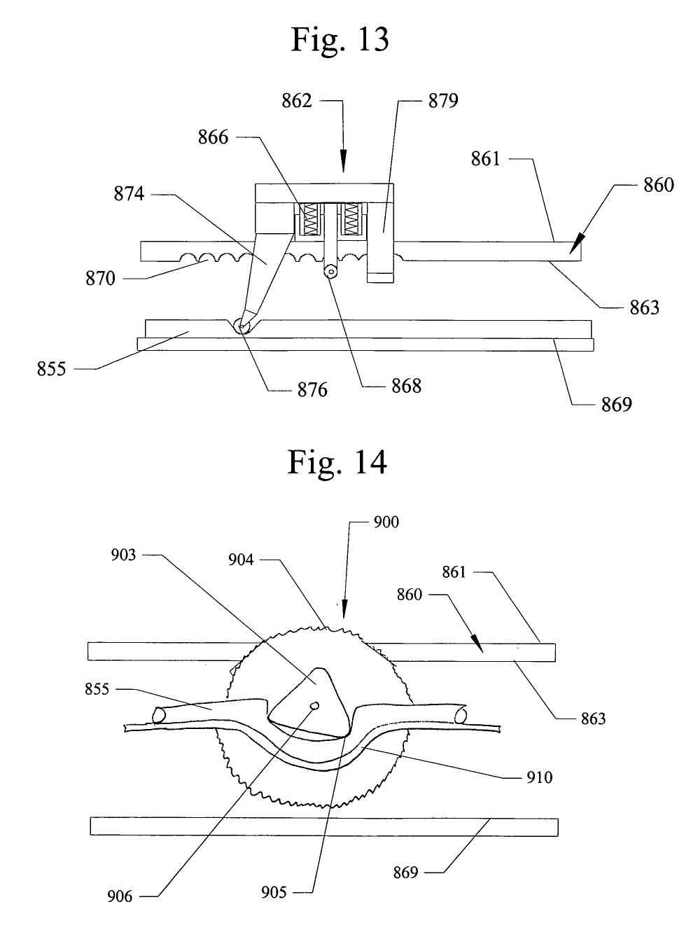

Some embodiments of the delivery device include a means for selectively injecting a desired amount of the IR-visible substance and/or drug. In the embodiment of FIGS. 8A-9B, a syringe having graduations on its outer surface serves as this means. However, other embodiments utilize different means. For example, two embodiments of such a means are shown in FIGS. 13 and 14, each of which operates in a manner similar to that of an art recognized peristaltic pump insofar as each includes a flexible tube 855 that filled with an IR visible substance or drug and compressed in order to push a desired amount of the IR visible substance and/or drug into a needle (not shown).

In the embodiment of FIG. 13, the body 860 of the delivery device includes a slot into which an actuator 862 is disposed. The actuator 862 includes a spring loaded engagement system 864 made up a pair of compression springs 866 that are dimensioned engage with the outside surface 861 of the body 860 proximate to the slot and exert an upward force on the actuator 862, a retaining member 868 that is dimensioned to mate with a plurality of detents 870 disposed in the inside surface 863 of the top 867 of body 860 proximate to the slot, and a stabilizer 879 that is dimensioned to engage the inside surface 863 of the top 867 of body 860 proximate to the slot when the retaining member 868 is mated with a detent 870. The actuator 862 also includes an extension arm 874 that extends downward into the body 860 and a roller 876 disposed at the end of the extension arm 874. The extension arm 874 and roller 876 are dimensioned to exert a compressive force upon the flexible tube 855 sufficient for the tube 855 to be collapsed between the roller 876 and the inside surface 863 of the bottom 869 of the body 860 when the actuator is fully depressed.

When the actuator 862 is unengaged, the springs 866 maintain the retaining member 868 in frictional engagement with one of the detents 870 and the stabilizer 879 in engagement with the inside surface 863 of the top 867 of body 860 proximate to the slot. When the actuator 862 is in this position, the roller 876 does not exert sufficient pressure upon the flexible tube 855 to collapse it. However, when a user pushes depresses the actuator, the retaining member 862 disengages from the detent 870 and the roller 876 exerts a compressive force upon the flexible tube 855 sufficient for the tube 855 to be collapsed between the roller 876 and the inside surface 863 of the bottom 869 of the body 860. The user then moves the actuator 862 forward a desired distance within the slot, causing a proportional amount of the IR visible substance or drug out of the tube 855 and into a needle (not shown). The desired distance of travel preferably corresponds to gradations along the slot that correspond to volumetric amounts of the fluid that have been dispensed based upon such movement. After the desired amount has been dispensed, the user releases the actuator 862 and the springs 866 again force the actuator 862 upward such that the engaging member 868 frictionally engages another one of the detents 870.

The inventor contemplates a number of different embodiments that utilize the same principles as are employed in the embodiment of FIG. 13. For example, in some embodiments, a rocker (not shown) attached to the stabilizer 879 and a single spring 866 may replace the captured springs 866 shown in FIG. 13. In other embodiments, the retaining member 868 is spring loaded rather than the entire actuator 862. In such an embodiment, the roller 876 is dimensioned to collapse the flexible tube 855 at all times and the advancement of the actuator 862 causes the retaining member 868 to follow the contour of the detents 870 while the cessation of such advancement causes the retaining member 868 to hold the actuator 862 in place. In such an embodiment, it is preferred that the actuator 862 also include a release mechanism that allows the roller 876 to be disengaged from the tube 855, allowing it to be moved backward in the slot in preparation for re-advancement. In other embodiments, the actuation is accomplished by an electrometrical linear or rotational actuator (not shown), that allows for ease of operation and provides very precise control of how much of the fluid is dispensed.

FIG. 14 shows another embodiment of a means 900 for selectively injecting a desired amount of the IR-visible substance and/or drug. This means 900 includes a triangular member 903 that is mounted on a central axle 906 in concentric relation with a thumb wheel 904. The triangular member 903 includes three contact surfaces 905 at its tips, which contacts and exert a compressive force upon the flexible tube 855 sufficient for the tube 855 be collapsed between the contact surface 905 and a backing member 910. As shown in FIG. 14, the backing member 910 is shaped to accept the flexible tube 855 and ensure that the tube 855 is collapsed by at least one contact surface 905 at all times in order to prevent an outflow of the IR visible substance or drug that fills the tube 855. In operation, the user will roll the thumb wheel 904 toward the needle (not shown) to force the fluid from the flexible tube 855 and through the needle and will roll the thumb wheel 904 away from the needle (not shown) to force the fluid from the needle and back through the flexible tube 855. It should be recognized that the ability of this embodiment of the means 900 to both dispense fluids through the needle and to back-flush the needle of the fluid is a distinct advantage.

As shown in FIG. 14, the bottom of the thumb wheel 904 is disposed within the body 860 of the device and the top of the thumb wheel 904 extends through a slot in the top 863 thereof. However, it is recognized that a slot could be included in the bottom 869 of the body 860 and that the bottom of the thumb wheel 904 could extend therethrough in a similar manner. Further, although the use of a separate backing member 910 is preferred, it is recognized that the inside surface 863 of the bottom 869 of the body 860 may be used in a manner similar to that of the embodiment of FIG. 13 to achieve similar results.

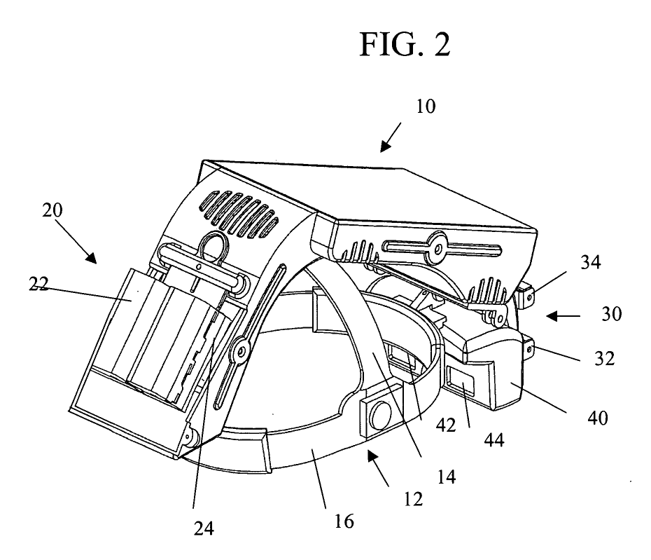

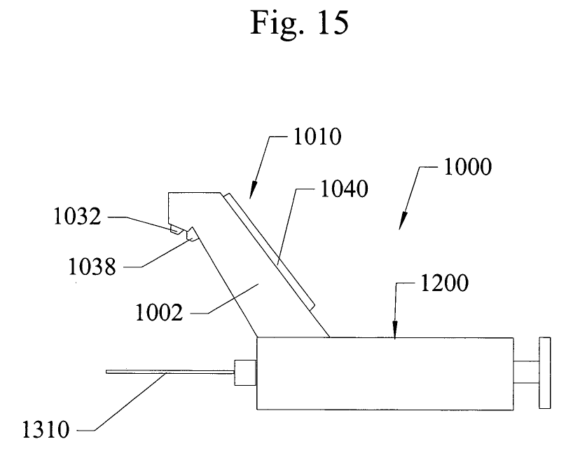

In some embodiments of the delivery system, the imaging system and the delivery system are integrated together. As shown in FIG. 15, the combined imaging and delivery system 1000 includes a delivery device 1200 having an extension 1002 that extends upward and forward towards the cannula 1310. An infrared emitter 1032 and camera 1038 are attached to the extension 1002 and are angled downward toward the tip of the cannula 1310 and a display 1040 is disposed upon the back side of the extension 1002. A computing unit (not shown) and power source (not shown) are disposed within the extension 1002 and preferably operate in a manner similar to the headset type embodiments described with reference to FIGS. 1 and 2.

In operation, the delivery system 1000 is aligned with the surface of a user's skin and the imaging system 1010 is powered on. The blood vessels are viewed through the display 1040 and the cannula 1310 is aligned therewith. The cannula 1310 is then inserted and the procedure performed in a manner similar to the embodiments described above.

It is noted that all components of the imaging system need not be included on the device. For example, the infrared emitter 1032, camera 1038, and computing unit may be mounted separately from the delivery device 1200 and communicate wirelessly with the display 1040 mounted on the delivery device 1200. Similarly, the display 1040 and computing unit may be separately mounted and the infrared emitter 1032 and camera 1038 mounted on the delivery device 1200. Finally, in some embodiments, an infrared emitter 1032 is the only component mounted on the delivery device 1200 and is used to provide enhanced localized illumination of the area to be viewed. Finally, it is recognized that the delivery device 1200 may include any of the features shown in the other embodiments described herein. Accordingly, the combined system should not be seen as being limited to the preferred embodiment shown and described in FIG. 15.

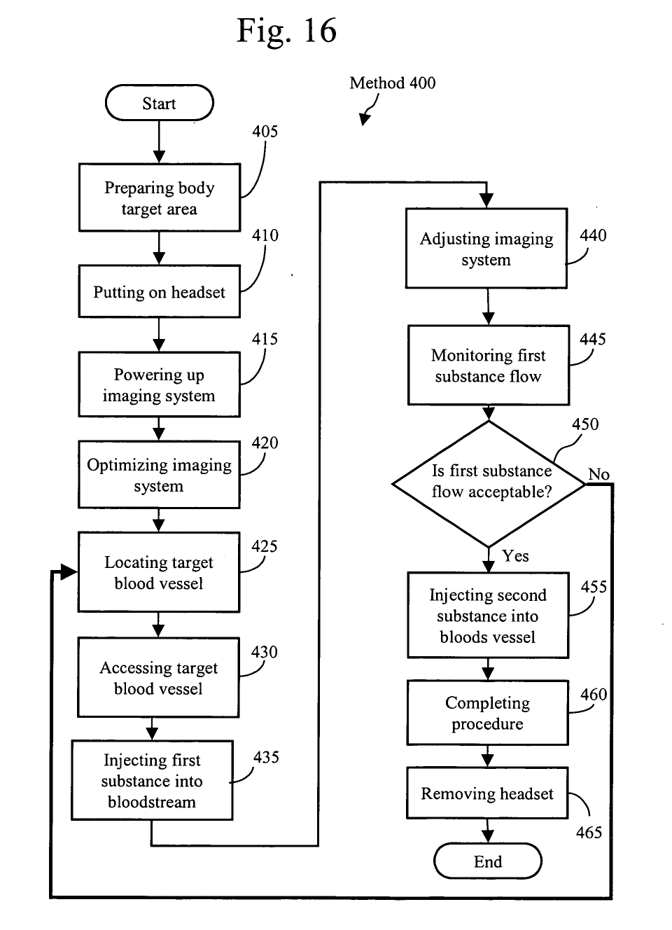

FIG. 16 illustrates a flow diagram of a preferred method 400 of delivering substances into blood vessels and observing the flow of a first of these substances to verify proper delivery of the a subsequent substance or substances. In the preferred method 400, the preferred imaging system 10, described above, is utilized. The preferred method 400 includes the steps of:

Step 405: Preparing Body Target Area

In this step, a user, such as a medical practitioner (e.g., doctor, nurse, or technician), prepares the patient's body target area for injection by using standard medical practices. This might include, for example, positioning the target body area (e.g., arm), applying a tourniquet, swabbing the target area with disinfectant, and palpating the target area. Method 400 then proceeds to step 410.

Step 410: Putting on the Headset 12

In this step, the user places the headset 12 on his/her head and adjusts head mount 16 for size, comfort, and a secure fit. Method 400 then proceeds to step 415.

Step 415: Powering Up the System

In this step, the user powers up the imaging system 10, by activating a switch controlling the power source 20. Method 400 proceeds to step 420.

Step 420: Optimizing the System

In this step, the user uses data input 255 to adjust various parameters of the imaging system 10, including specifying the appropriate digital signal processor 122 algorithms (according to, for example, the patient's body type, pigmentation, age), intensity levels of the infrared emitters 32, 34, and parameters for the images to be viewed on the display 40. Method 400 then proceeds to step 425.

It should be noted that Steps 410, 415, and 420 may be performed in any order, e.g., the user may power up the imaging system 10 and optimize it, prior to putting it on. Further, it is recognized that optimizing step 420 may be eliminated altogether, with settings of the imaging system 10 being preset at the factory.

Step 425: Locating Target Blood Vessel

In this step, the user searches non-invasively for the desired target blood vessel(s) (e.g., vein, artery, or capillary bed), by directing the incident light 215 from the infrared emitters 32, 34 on the body target area, viewing the target area on display 40, and focusing the camera lens 240 on the skin surface 225. As viewed on display 40, the target blood vessel(s) will be visually enhanced, i.e., appear different from the surrounding tissue, which enables the user to insert the cannula 310 of the delivery device 200 more accurately and rapidly, in order to gain IV access for injection. Because of the hands-free operation of the preferred imaging system 10, the user is free to handle the body target area with both hands, for stability, further palpation, and cleansing, for example. Using the imaging system 10 in a bifocal mode, the user may look down from display 40 to see the body target area as it appears under normal, non-enhanced conditions. Second lens 260 adjusts the image displayed on display 40 for depth perception differences between the enhanced image and the image viewed directly by the user. Method 400 proceeds to step 430.

Step 430: Accessing Target Blood Vessel

In this step, the user, by utilizing either his/her naked eye or the enhanced image appearing on display 40, pulls the patient's skin tightly over the target blood vessel located in step 425 and aligns the cannula 310 directly over and parallel to the target blood vessel, and pierces skin surface 225 with the cannula 310 of the delivery device 200. The user then advances the cannula 310. In embodiments in which an IR-visible substance is applied to the cannula sheathing 320, or formed integral thereto, cannula sheathing 320 becomes visible via display 40, which allows user to determine the accuracy of the needle placement. U.S. Patent Applications US2004/0019280, US2003/0187360, and US2002/0115922 fully describe a system in which an IR-opaque or IR-reflective substance or pattern is applied to cannula sheathing 320, which makes the travel path of cannula sheathing 320 clearly visible to user via display 40, so that user may gauge its position and travel path more accurately. Alternatively, the cannula tip 330 may be doped with an IR-opaque or IR-reflective substance or pattern, which makes the travel path of cannula tip 330 clearly visible to the user via display 40, so that user may gauge its position and travel path more accurately. By using the enhanced image of the target blood vessel and the cannula 310 displayed via display 40, the user is able to access the appropriate blood vessel more accurately and rapidly and ensure that the cannula 310 is advanced the desired distance.

Method 400 proceeds to step 435.

Step 435: Delivering a First Visible Substance into the Blood Vessel

In this step, the user introduces a first substance into the target blood vessel by injecting it through the cannula 310 of the delivery device 200. The first substance is preferably an IR-visible substance, such as indocyanine green, although any substance commonly delivered into a blood vessel may be delivered. The amount of the first substance introduced depends on the application and monitoring period of method 400 and, therefore, is determined by the medical practitioner. Method 400 proceeds to step 440.

Step 440: Adjusting the System

In this optional step, the user uses data input 255 to optimize the imaging system 10 in order to better view the first substance introduced in step 335. This may include an adjustment of the algorithms performed by the digital signal processor 122, intensity levels and/or wavelengths of light emitted by the infrared emitters 32, 34, and parameter of the display 40, such as contrast and focal length, or other parameters of the imaging system 10. In some embodiments, this step involves adjusting the system based upon characteristics of the first substance delivered in step 435 such that the system is optimized for the particular first substance. Method 400 proceeds to step 445.

Step 445: Examining Flow Patterns

In this step, the user, utilizing the enhanced image appearing on display 40, examines the flow patterns of the first substance introduced in step 435. As viewed on display 40, the first substance will be visually enhanced, i.e., appear different from the surrounding tissues and structures. Typically, this step involves examining the images on the display 40 to detect whether (1) the first substance leaks outside of the target blood vessel, (2) the first substance flows in the intended direction within the target blood vessel, and (3) the first substance flows to the proper destination within the patient's bloodstream. In some embodiments, the flow pattern sequences are recorded on data storage 245 and reviewed on display 40 (or external device) at a later time. Upon playback, digital signal processor 122 may be adjusted to alter flow pattern sequences by speeding the sequences up, slowing the sequences down, or otherwise modifying flow pattern sequences, in order to aid the user in viewing and diagnosing. Method 400 proceeds to step 450.

Step 450: Determining Whether the Flow of the First Substance is Acceptable

In this decision step, the user determines whether the flow of the IR-opaque substance within the patient's bloodstream is acceptable based upon the result of the examining step. If yes, method 400 proceeds to step 455. If no, user withdraws cannula 320 and method 400 loops back to step 425.

Step 455: Injecting a Second Substance into Bloodstream

In this step, user injects a predetermined amount of a second substance (e.g., chemotherapeutic drugs, saline solutions, etc.) through the cannula 320 of the delivery device 200. In some embodiments, this is accomplished by means of a standard hypodermic needle that has been pre-loaded with the drug. In these embodiments, the substance flows from cannula housing 340, into cannula sheathing 320, out of cannula tip 330, and into the target blood vessel. In embodiments utilizing other delivery devices, this injection step is performed in the manner described above in connection with the particular embodiment of the delivery device that is utilized. Method 400 proceeds to step 460.

Step 460: Completing Procedure

In this step, the user completes the injection by using standard medical practices. This may include, for example, withdrawing the cannula and cleansing the injection area, or releasing a tourniquet and attaching IV tubing to cannula housing 340. Method 400 proceeds to step 465.

Step 465: Removing the Headset 12

In this step, the user removes the headset 12 from his/her head and powers off the imaging system 10. Alternatively, the user prepares additional patients/body target areas for imaging and injection. Method 400 ends.

As noted above, the delivery system of the present invention is not limited to those embodiments utilizing the preferred imaging system 10, but rather may be performed using any imaging system that includes at least one infrared emitter and a power source. Due to the injection of a highly visible substance within the blood vessel, and the fact that the step 445 of examining flow patterns does not require that real time images be provided to the display, the imaging system used to perform the method may not enhance images, or provide images to the display in substantially real time. Further, in embodiments in which only an infrared emitter is used to transilluminate a blood vessel, no images are provided at all. Therefore, in these embodiments, steps 405, 410, 420, and 440 may be omitted, and step 425 may be performed after the blood vessel has been accessed and the first substance has been injected.

Method 400 may be used for a single drug delivery, or may be used multiple times. In cases for which multiple deliveries are made, the method may further include the step of flushing residual drug from the cannula 310 before repeating steps 425-465 of the method 400. However, where the method is performed utilizing an embodiment of the delivery system that comprises a multiple needle delivery device, this flushing step may be omitted.

Although the present invention has been described in considerable detail with reference to certain preferred versions thereof, other versions would be readily apparent to those of ordinary skill in the art. Therefore, the spirit and scope of the appended claims should not be limited to the description of the preferred versions contained herein.

What is claimed is:

1. A delivery device for delivering a first substance and a second substance into a blood vessel, said delivery device comprising:
a body comprising a first end, a second end, an outer surface;
a first substance reservoir disposed within said body;
at least one cannula extending from said first end of said body, wherein said cannula comprises a cannula tip having a cannula opening therethrough and a cannula sheathing defining an interior passage, and wherein said interior passage of one of said at least one cannula is in fluid communication with said first substance reservoir;
a means for delivering the first substance from said first substance reservoir through one of said at least one cannula; and
a means for delivering the second substance through one of said at least one cannula.

2. The delivery device of claim 1, wherein said means for delivering the second substance comprises a drug port extending from said outside surface of said body, wherein said drug port is dimensioned to allow passage of a hypodermic needle therethrough and is in fluid communication with one of said at least one cannula such that said substance may be delivered from said hypodermic needle through said drug port and said cannula.

3. The delivery device of claim 2 wherein said first substance reservoir comprises a substantially cylindrical bore extending into said body from said second end of said body, and wherein said means for delivering said first substance from said first substance reservoir through one of said at least one cannula comprises a plunger dimensioned to mate with said cylindrical bore and push the first substance disposed within said first substance reservoir through said cannula.

4. The delivery device of claim 3 wherein said plunger further comprises a through-hole dimensioned to allow a catheter needle to be disposed therethrough and a sealing means for sealing said plunger about said catheter needle such that the first substance cannot leak through said through-hole when said catheter needle is disposed therethrough, wherein said interior passage of said cannula is in concentric relation with said through-hole and is dimensioned to allow the catheter needle to be inserted therein, and wherein said cannula opening of said cannula tip is dimensioned to prevent passage of said catheter needle therethrough.

5. The delivery device of claim 2 wherein said first substance reservoir comprises a hollow portion of said body, and wherein said means for delivering said first substance from said first substance reservoir through one of said at least one cannula comprises a pump actuator extending from said second end of said body, a one-way valve in communication with said pump actuator, and an internal bladder disposed within said first substance reservoir proximate said second end of said body, wherein said pump actuator is adapted to pump air through said one-way valve to inflate internal bladder such that the first substance is forced from said substance reservoir through said cannula.

6. The delivery device of claim 1 wherein said first substance reservoir comprises a hollow portion of said body, and wherein said means for delivering said first substance from said first substance reservoir through one of said at least one cannula comprises a pump actuator extending from said second end of said body, a one-way valve in communication with said pump actuator, and an internal bladder disposed within said first substance reservoir proximate said second end of said body, wherein said pump actuator is adapted to pump air through said one-way valve to inflate internal bladder such that the first substance is forced from said first substance reservoir through said cannula.

7. The delivery device of claim 1 wherein said first substance reservoir comprises a substantially cylindrical bore extending into said body from said second end of said body, and wherein said means for delivering said first substance from said first substance reservoir through one of said at least one cannula comprises a first plunger dimensioned to mate with said cylindrical bore and push the first substance disposed within said first substance reservoir through said cannula.

8. The delivery device of claim 7 wherein said means for delivering a second substance through one of said at least one cannula comprises a second substance reservoir disposed within said first plunger and in fluid communication with one of said at least one cannula, and a means for delivering said second substance from said second substance reservoir through one of said at least one cannula.

9. The delivery device of claim 8 further comprising a flexible tube in fluid communication with said second substance reservoir and said cannula, wherein said second substance reservoir comprises a substantially cylindrical bore extending into said first plunger, and wherein said means for delivering said second substance from said first substance reservoir through one of said at least one cannula comprises a second plunger dimensioned to mate with said cylindrical bore and push the second substance disposed within said second substance reservoir through said flexible tube and said cannula.

10. The delivery device of claim 8 further comprising safety means for preventing one of said first substance and said second substance from being delivered through said cannula before another of said first substance and said second substance has been delivered through said cannula.

11. The delivery device of claim 1 further comprising a second substance reservoir in fluid communication with one of said at least one cannula;
wherein said first substance reservoir comprises a tube filled with the first substance and said second substance reservoir comprises a tube filled with the second substance; and
wherein said body comprises at least two mating bores in which said first substance reservoir and said second substance reservoir are disposed and secured;
wherein said means for delivering a first substance from said first substance reservoir through one of said at least one cannula comprises a first selector disposed upon said body and adapted to control the delivery of the first substance from said first substance reservoir through said at least one cannula; and
wherein said means for delivering a second substance through one of said at least one cannula comprises a second selector disposed upon said body and adapted to control the delivery the second substance from said second substance reservoir through said at least one cannula.

12. The delivery device of claim 11 wherein said first substance reservoir comprises a pressurized tube filled with the first substance and said second substance reservoir comprises a pressurized tube filled with the second substance.

13. The delivery device of claim 11 further comprising at least one actuator adapted to deliver at least one of said first substance and said second substance through said at least one cannula;
wherein said means for delivering a first substance from said first substance reservoir through one of said at least one cannula further comprises one of said at least one actuator; and
wherein said means for delivering a first substance from said second substance reservoir through one of said at least one cannula further comprises one of said at least one actuator.

14. The delivery device of claim 13 wherein said at least one actuator comprises a first actuator and a second actuator;
wherein said means for delivering the first substance from said first substance reservoir through one of said at least one cannula comprises said first actuator in communication with said first substance reservoir; and
wherein said means for delivering the second substance from said second substance reservoir through one of said at least one cannula further comprises said second actuator.

15. The delivery device of claim 11 further comprising safety means for preventing one of said first substance and said second substance from being delivered through said at least one cannula before another of said first substance and said second substance has been delivered through said at least one cannula.

16. The delivery device of claim 11 wherein said at least one cannula comprises a first cannula and a second cannula, wherein said first substance reservoir is in fluid communication with said first cannula and wherein said second substance reservoir is in fluid communication with said second cannula.

17. The delivery device of claim 11 further comprising a third substance reservoir in fluid communication with one of said at least one cannula;
wherein said third substance reservoir comprises a tube filled with a third substance;
wherein said body comprises at least three mating bores in which said first substance reservoir, said second substance reservoir, and said third substance reservoir are disposed and secured, and at least three selectors adapted to control the delivery of the first substance from said first substance reservoir through said at least one cannula, the second substance from said second substance reservoir through said at least one cannula, and the second substance from said second substance reservoir through said at least one cannula.

18. The delivery device of claim 17 wherein said first substance reservoir is an IR-visible substance reservoir filled with an IR-visible substance, wherein said second substance reservoir is a drug reservoir filled with a drug, and said third reservoir is a flushing reservoir filled with a flushing substance; and wherein said device further comprises a safety means for controlling the operation of the selectors such that the drug may not be delivered before a first amount of the IR-visible substance has been delivered, and such that second amount of the IR-visible substance may not be delivered until the flushing substance has been delivered through said at least one cannula.

19. The delivery device of claim 1 wherein said at least one cannula comprises a first cannula and a second cannula, wherein said first substance reservoir is in fluid communication with said first cannula and wherein said means for delivering a second substance through one of said at least one cannula is in fluid communication with said second cannula.

20. The delivery device of claim 19 wherein said means for delivering a second substance through one of said at least one cannula further comprises a second substance reservoir in fluid communication with said second cannula.

21. The delivery device of claim 20 wherein said means for delivering a first substance through said at least one cannula comprises a first actuator in communication with said first substance reservoir; and
wherein said means for delivering a second substance through one of said at least one cannula further comprises a second actuator in communication with a second substance reservoir.

22. The delivery device of claim 21 wherein at least one of said first actuator and said second actuator is a plunger.

23. The delivery device of claim 19 wherein said means for delivering a second substance through one of said at least one cannula further comprises a second substance reservoir in fluid communication with at least one cannula.

24. The delivery device of claim 23 wherein said means for delivering a first substance through said at least one cannula comprises a first actuator in communication with said first substance reservoir; and
wherein said means for delivering a second substance through one of said at least one cannula further comprises a second actuator in communication with a second substance reservoir.

25. The delivery device of claim 24 wherein at least one of said first actuator and said second actuator is a plunger.

26. The delivery device of claim 1 wherein said means for delivering the first substance from said first substance reservoir through one of said at least one cannula comprises a means for selectively delivering a desired amount of the first substance from said first substance reservoir through one of said at least one cannula.

27. The delivery device of claim 26 wherein said means for selectively delivering a desired amount of the first substance from said first substance reservoir through one of said at least one cannula comprises a substantially flexible tube, a means for collapsing a portion of said substantially flexible tube and an means for moving said means for collapsing a portion of said substantially flexible tube toward said at least one cannula.

28. A delivery system for accurately delivering a substance into a blood vessel, said delivery system comprising:
a delivery device for delivering a first substance and a second substance into a blood vessel, wherein one of said first substance and said second substance is an IR-visible substance, said delivery device comprising:
a body comprising a first end, a second end, an outer surface;
a first substance reservoir disposed within said body;
at least one cannula extending from said first end of said body, wherein said cannula comprises a cannula tip having a cannula opening therethrough and a cannula sheathing defining an interior passage, and wherein said interior passage of one of said at least one cannula is in fluid communication with said first substance reservoir;
a means for delivering the first substance from said first substance reservoir through one of said at least one cannula; and
a means for delivering the second substance through one of said at least one cannula; and
an imaging system comprising:
at least one infrared emitter configured to illuminate a region under a surface of skin with waves of infrared light;
an infrared detector configured to accept waves of infrared light reflected from the region under the surface of the skin, said infrared detector comprising an output for outputting a signal corresponding to image data;
a computing unit comprising an input for accepting said image data from said infrared detector, and an output for outputting images corresponding to said image data;
a display device for inputting said images from said output of said computing unit and displaying said images; and
a power source in electrical communication with said infrared emitter, said infrared detector, said computing unit and said display device;
wherein a user disposes said at least one cannula of the delivery device within a blood vessel located beneath the surface of the skin, delivers the IR-visible substance into the blood vessel, views images of the IR-visible substance on the display of the imaging system to examine a flow pattern of the IR-visible substance and verify that said at least one cannula is properly disposed within a desired blood vessel, and delivers the second substance through one of said at least one cannula into the blood vessel.

29. The delivery system of claim 28 wherein said means for delivering the second substance of said delivery device comprises a drug port extending from said outside surface of said body, wherein said drug port is dimensioned to allow passage of a hypodermic needle therethrough and is in fluid communication with one of said at least one cannula such that said substance may be delivered from said hypodermic needle through said drug port and said cannula.

30. The delivery system of claim 29 wherein said first substance reservoir of said delivery device comprises a substantially cylindrical bore extending into said body from said second end of said body, and wherein said means for delivering said first substance from said first substance reservoir through one of said at least one cannula comprises a plunger dimensioned to mate with said cylindrical bore and push the first substance disposed within said first substance reservoir through said cannula.

31. The delivery system of claim 30 wherein said plunger further comprises a through-hole dimensioned to allow a catheter needle to be disposed therethrough and a sealing means for sealing said plunger about said catheter needle such that the first substance cannot leak through said through-hole when said catheter needle is disposed therethrough, wherein said interior passage of said cannula is in concentric relation with said through-hole and is dimensioned to allow the catheter needle to be inserted therein, and wherein said cannula opening of said cannula tip is dimensioned to prevent passage of said catheter needle therethrough.

32. The delivery system of claim 29 wherein said first substance reservoir of said delivery device comprises a hollow portion of said body, and wherein said means for delivering said first substance from said first substance reservoir through one of said at least one cannula comprises a pump actuator extending from said second end of said body, a one-way valve in communication with said pump actuator, and an internal bladder disposed within said first substance reservoir proximate said second end of said body, wherein said pump actuator is adapted to pump air through said one-way valve to inflate internal bladder such that the first substance is forced from said substance reservoir through said cannula.

33. The delivery system of claim 28 wherein said first substance reservoir of said delivery device comprises a hollow portion of said body, and wherein said means for delivering said first substance from said first substance reservoir through one of said at least one cannula comprises a pump actuator extending from said second end of said body, a one-way valve in communication with said pump actuator, and an internal bladder disposed within said first substance reservoir proximate said second end of said body, wherein said pump actuator is adapted to pump air through said one-way valve to inflate internal bladder such that the first substance is forced from said first substance reservoir through said cannula.

34. The delivery system of claim 28 wherein said first substance reservoir of said delivery device comprises a substantially cylindrical bore extending into said body from said second end of said body, and wherein said means for delivering said first substance from said first substance reservoir through one of said at least one cannula comprises a first plunger dimensioned to mate with said cylindrical bore and push the first substance disposed within said first substance reservoir through said cannula.

35. The delivery system of claim 34 wherein said means for delivering a second substance through one of said at least one cannula comprises a second substance reservoir disposed within said first plunger and in fluid communication with one of said at least one cannula, and a means for delivering said second substance from said second substance reservoir through one of said at least one cannula.

36. The delivery system of claim 35 wherein said delivery device further comprises a flexible tube in fluid communication with said second substance reservoir and said cannula, wherein said second substance reservoir comprises a substantially cylindrical bore extending into said first plunger, and wherein said means for delivering said second substance from said first substance reservoir through one of said at least one cannula comprises a second plunger dimensioned to mate with said cylindrical bore and push the second substance disposed within said second substance reservoir through said flexible tube and said cannula.

37. The delivery system of claim 35 wherein said delivery device further comprises safety means for preventing one of said first substance and said second substance from being delivered through said cannula before another of said first substance and said second substance has been delivered through said cannula.

38. The delivery device of claim 28 wherein said delivery device further comprises a second substance reservoir in fluid communication with one of said at least one cannula;
wherein said first substance reservoir comprises a tube filled with the first substance and said second substance reservoir comprises a tube filled with the second substance; and
wherein said body comprises at least two mating bores in which said first substance reservoir and said second substance reservoir are disposed and secured;
wherein said means for delivering a first substance from said first substance reservoir through one of said at least one cannula comprises a first selector disposed upon said body and adapted to control the delivery of the first substance from said first substance reservoir through said at least one cannula; and
wherein said means for delivering a second substance through one of said at least one cannula comprises a second selector disposed upon said body and adapted to control the delivery the second substance from said second substance reservoir through said at least one cannula.

39. The delivery system of claim 38 wherein said first substance reservoir comprises a pressurized tube filled with the first substance and said second substance reservoir comprises a pressurized tube filled with the second substance.

40. The delivery system of claim 39 wherein said delivery device further comprises at least one actuator adapted to deliver at least one of said first substance and said second substance through said at least one cannula;
wherein said means for delivering a first substance from said first substance reservoir through one of said at least one cannula further comprises one of said at least one actuator; and
wherein said means for delivering a first substance from said second substance reservoir through one of said at least one cannula further comprises one of said at least one actuator.

41. The delivery system of claim 40 wherein said at least one actuator comprises a first actuator and as second actuator;
wherein said means for delivering the first substance from said first substance reservoir through one of said at least one cannula comprises said first actuator in communication with said first substance reservoir; and
wherein said means for delivering the second substance from said second substance reservoir through one of said at least one cannula further comprises said second actuator.

42. The delivery system of claim 38 wherein said delivery device further comprises safety means for preventing one of said first substance and said second substance from being delivered through said at least one cannula before another of said first substance and said second substance has been delivered through said at least one cannula.

43. The delivery system of claim 38 wherein said at least one cannula comprises a first cannula and a second cannula, wherein said first substance reservoir is in fluid communication with said first cannula and wherein said second substance reservoir is in fluid communication with said second cannula.

44. The delivery system of claim 38 wherein said delivery device further comprises a third substance reservoir in fluid communication with one of said at least one cannula;
wherein said third substance reservoir comprises a tube filled with a third substance;
wherein said body comprises at least three mating bores in which said first substance reservoir, said second substance reservoir, and said third substance reservoir are disposed and secured, and at least three selectors adapted to control the delivery of the first substance from said first substance reservoir through said at least one cannula, the second substance from said second substance reservoir through said at least one cannula, and the second substance from said second substance reservoir through said at least one cannula.

45. The delivery system of claim 44 wherein said first substance reservoir is an IR-visible substance reservoir filled with the IR-visible substance, wherein said second substance reservoir is a drug reservoir filled with a drug, and said third reservoir is a flushing reservoir filled with a flushing substance; and wherein said device further comprises a safety means for controlling the operation of the selectors such that the drug may not be delivered before a first amount of the IR-visible substance has been delivered, and such that second amount off the IR-visible substance may not be delivered until the flushing substance has been delivered through said at least one cannula.

46. The delivery system of claim 28 wherein said at least one cannula of said delivery device comprises a first cannula and a second cannula, wherein said first substance reservoir is in fluid communication with said first cannula and wherein said means for delivering a second substance through one of said at least one cannula is in fluid communication with said second cannula.

47. The delivery system of claim 46 wherein said means for delivering a second substance through one of said at least one cannula further comprises a second substance reservoir in fluid communication with said second cannula.

48. The delivery system of claim 47 wherein said means for delivering a first substance through said at least one cannula comprises a first actuator in communication with said first substance reservoir; and
wherein said means for delivering a second substance through one of said at least one cannula further comprises a second actuator in communication with a second substance reservoir.

49. The delivery system of claim 28 wherein at least one substance for enhancing a visibility of said cannula by said imaging system is disposed upon at least one of said cannula tip and said cannula sheathing, wherein said at least one substance enhances a visibility of said cannula by said imaging system when compared with a visibility of said cannula without said substance disposed thereon.

50. The delivery system of claim 46 wherein said means for delivering a second substance through one of said at least one cannula further comprises a second substance reservoir in fluid communication with at least one cannula.

51. The delivery system of claim 50 wherein said means for delivering a first substance through said at least one cannula comprises a first actuator in communication with said first substance reservoir; and
wherein said means for delivering a second substance through one of said at least one cannula further comprises a second actuator in communication with a second substance reservoir.

52. The delivery system of claim 51 wherein at least one of said first actuator and said second actuator is a plunger.

53. The delivery system of claim 28 wherein said computing unit of said imaging system further comprises a memory and means for enhancing and outputting result images in which enhanced images of blood vessels are shown within images of the region under the surface of the skin, and wherein said images corresponding to said image data are said result images.

54. The delivery system of claim 53 wherein said imaging system further comprises a headset, wherein said at least one infrared emitter, said infrared detector, said computing unit, said display, and said power source of said imaging system are attached to said headset, and wherein said display is disposed such that a user is able to view both said display and the surface of the skin without removing said headset.

55. The delivery system of claim 54 wherein said infrared detector of said imaging system comprises a CMOS camera adapted to generate digital data corresponding to said waves of infrared light reflected from the subcutaneous blood vessels located in the region under the surface of the skin.

56. The delivery system of claim 55 wherein said imaging system further comprises a camera lens disposed between the surface of the skin and said CMOS camera.

57. The delivery system of claim 54 wherein said display of said imaging system comprises at least one LCD screen.

58. The delivery system of claim 57 wherein said imaging system further comprises an optical lens disposed between said LCD screen and an eye of a user.

59. The delivery system of claim 54 wherein said computing unit of said imaging system further comprises an interface and wherein said means for enhancing and outputting result images comprises a digital signal processing unit.

60. The delivery system of claim 59 wherein said imaging system further comprises a data input in communication with said digital signal processing unit through said interface.

61. The delivery system of claim 52 wherein said imaging system further comprises a data input, wherein said means for enhancing and outputting result images comprises a digital signal processing unit and wherein said data input is in communication with said digital signal processing unit.

62. The delivery system of claim 28 wherein at least one of said at least one infrared emitter, said infrared detector, said computing unit, said display device, and said power source of said imaging system are attached to said delivery device.

63. The delivery system of claim 28 wherein said means for delivering the first substance from said first substance reservoir through one of said at least one cannula of said delivery device comprises a means for selectively delivering a desired amount of the first substance from said first substance reservoir through one of said at least one cannula.

64. The delivery device of claim 63 wherein said means for selectively delivering a desired amount of the first substance from said first substance reservoir through one of said at least one cannula of said delivery device comprises a substantially flexible tube, a means for collapsing a portion of said substantially flexible tube and an means for moving said means for collapsing a portion of said substantially flexible tube toward said at least one cannula.

65. A method for delivering a second substance into blood vessels using a delivery device and observing the flow of a first substance with the aid of an imaging system to verify proper delivery of the second substance, wherein the imaging system comprises at least one infrared emitter, an infrared detector, a computing unit in communication with the infrared detector, a display in communication with the computing unit, and a power source, wherein the delivery device comprises a body comprising a first end, a second end, an outer surface, a first substance reservoir disposed within the body, at least one cannula extending from the first end of said body, wherein the cannula comprises a cannula tip having a cannula opening therethrough and a cannula sheathing defining an interior passage, and wherein the interior passage of one of the at least one cannula is in fluid communication with the first substance reservoir, a means for delivering the first substance from the first substance reservoir through one of the at least one cannula, and a means for delivering the second substance through one of said at least one cannula; wherein said method comprises the steps of:
preparing a body target area;
supplying power from the power source to the infrared emitter, infrared detector, computing unit, and display of the imaging system, such that infrared light is emitted by the infrared emitter, reflected infrared light is received by the infrared detector and converted into signals sent to the computing unit, the computing unit accepts the signals and outputs image data to the display, and the display displays the images;
accessing a target blood vessel;
introducing the first substance into the target blood vessel;
locating the target blood vessel such that images of the target blood vessel are captured by the infrared detector and displayed on the display;
examining a flow of the first substance through the target blood vessel by viewing the images of the target blood vessel on the display of the imaging system;
determining whether the flow of the first substance flow is acceptable; and
delivering the second substance into the target blood vessel.

66. The method of claim 65 wherein said step of examining flow patterns comprises examining images displayed on the display to determine the presence of a leakage through the target blood vessel by observing the first substance flowing outside of the target blood vessel.

67. The method of claim 65 wherein said step of examining flow patterns comprises examining images displayed on the display to determine whether the first substance flows in an intended direction within the target blood vessel.

68. The method of claim 65 wherein said step of examining flow patterns comprises examining images displayed on the display to determine whether and the first substance flows to the proper destination within the patient's bloodstream.

69. The method of claim 65 wherein the computing unit of the imaging system enhances images of the target blood vessel before outputting the images to the display;
wherein the locating step is performed before the accessing step; and
wherein said accessing step comprises the step of viewing an enhanced image of the target blood vessel on the display of the imaging system and piercing the target blood vessel with the aid of the enhanced image.

70. The method of claim 69 wherein said locating step comprises the steps of:
directing incident light from the infrared emitters on a target area of a surface of a skin; and
viewing the enhanced image of blood vessels located beneath the target area on the display.

71. The method of claim 70 wherein the display of the imaging system comprises an optical lens disposed between the display and an eye of a user and wherein said locating step further comprises the steps of:
directly viewing an image on the target area of the skin; and
adjusting the optical lens to correct the enhanced image displayed on the display for depth perception differences between the enhanced image and a directly viewed image of the target area of the skin.

72. The method of claim 70 wherein said step of locating a target blood vessel further comprises the steps of:
directly viewing an image on the target area of the skin;
adjusting the display to correct the enhanced image displayed on display for depth perception differences between the enhanced image and a directly viewed image of the target area of the skin.

73. The method of claim 69 further comprising the step of optimizing the imaging system, wherein the computing unit comprises a digital signal processor and a memory, wherein the imaging system comprises a data input, and wherein said step of optimizing the imaging system comprises the step of using the data input to specify an enhancement algorithm stored in memory to be used by the digital signal processor to generate the enhanced image.

74. The method of claim 73 wherein said step of optimizing the system further comprises the step of selecting an enhancement algorithm based upon a factor selected from a group consisting of a body type, pigmentation, age of the patient, and characteristics of the first substance introduced into the target blood vessel.

75. The method of claim 73 wherein said step introducing a first substance into the target blood vessel comprises introducing an IR-visible substance into the target blood vessel, and wherein said step of optimizing the system further comprises the step of selecting an enhancement algorithm based upon and characteristics of the IR-visible substance.

76. The method of claim 73 wherein said step of optimizing the system further comprises the step of using said data input to adjust at least one of an intensity level of the at least one infrared emitter and a wavelength of infrared light emitted by said at least one infrared emitter.

77. The method of claim 65 wherein the imaging system further comprises a headset to which the infrared emitter, infrared detector, computing unit, and display are attached, and wherein said method further comprises the step of disposing the headset on a head of a user.

78. The method of claim 65 wherein the imaging system further comprises data storage means for storing multiple enhanced images and wherein said method further comprises the step of recording a sequence of enhanced images showing a flow pattern on the data storage means.

79. The method of claim 78 wherein the computing unit of the imaging system comprises a digital signal processor programmed with an algorithm to adjust the playback of the sequence of enhanced images stored in the data storage means.

80. The method of claim 65 further comprising the step of flushing the interior passage of the cannula after the step of injecting the second substance into the blood vessel.
